(19) United States
(12) Patent Application Publication
(10) Pub. No.: US 2019/0000667 A1
(43) Pub. Date: Jan. 3, 2019

(54) CONNECTABLE CATHETER SYSTEM

(71) Applicant: UROGEN PHARMA LTD., Ra'anana (IL)

(72) Inventors: Yosh DOLLBERG, Ra'anana (IL); Gil HAKIM, Ra'anana (IL); Nadav MALCHI, Ra'anana (IL)

(73) Assignee: Urogen Pharma Ltd., Ra'anana (IL)

(21) Appl. No.: 16/065,923

(22) PCT Filed: Dec. 29, 2016
(86) PCT No.: PCT/IL2016/051400

§ 371 Date: Jun. 25, 2018

Related U.S. Application Data
(60) Provisional application No. 62/272,456, filed on Dec. 29, 2015.

(51) Int. Cl. A61F 7/12; A61B 18/08; A61B 18/14; A61M 25/00; A61N 7/02

(52) U.S. Cl. CPC A61F 7/12; A61B 18/08; A61M 31/00; A61M 25/0017; A61N 7/022; A61B 18/1492

(57) ABSTRACT

There are provided a connectable catheter system, device and methods of use thereof. The connectable catheter system, comprising: an intermediary catheter and a reconnectable indwelling stent wherein the intermediary catheter and the indwelling stent are configured to connect, within a subject body to form a continuous fluid conduit between the intermediary catheter and the reconnectable indwelling stent and to further allow controlled release of substances and/or heating of internal target regions.

### FIELD OF THE INVENTION

The present invention relates to a connectable catheter system, device and methods of use thereof, wherein the catheter is configured to connect within a subject body and to allow sustained release of therapeutic substances and/or heating of target regions.

### BACKGROUND OF THE INVENTION

In the field of medical devices, a catheter is in essence a tube that can be inserted into the body to transfer fluids into and out of a human The catheter's size, shape and composition may be adjusted for its use, for example, for cardiovascular, urological, gastrointestinal, neurovascular, and ophthalmic applications. Catheters can be inserted into a body cavity, duct, or vessel. Functionally, they may allow drainage, administration of fluids, access by surgical instruments, and perform a wide variety of other medical procedures depending on the type of catheter. Depending on the application, a catheter is usually transiently inserted into the target organ and removed once the procedure is completed. A catheter that is left inside the body, either temporarily or permanently, is generally referred to as an indwelling catheter or stent.

Ureteral catheters, for example, are inserted through the meatus, through the bladder and ureter and advanced until the catheter extends from outside the body to the renal pelvis, for example, in order to deliver medications to the upper urinary tract. Due to severe risk of infection, ureteral catheters are mostly removed from the urinary tract immediately following the delivery of medication. If an additional installation of medication is required subsequently, an additional ureteral catheter is inserted for that purpose.

Ureteral stents (indwelling catheters) are deployed into the ureter and extend from the urinary bladder to the renal pelvis. They are used to dilate the ureter, for example, in order to facilitate the passage of urine and debris from the kidney and ureter to the bladder in cases of ureteral obstructions such as stones, strictures, edema, fistula, tumors, retro-peritoneal fibrosistumors, and hydronephrosis. Indications for use of a ureteral stent include such uses as: (I) as adjunct to such surgical procedures as extracorporeal shock wave lithotripsy (ESWL), percutaneous nephrolithotomy (PCNL), ureteroscopy, endopylometry, ureteral surgery, ureter injury, renal transplantation, upper tract urothelial carcinoma (UTUC) and conservative treatment of genitourinary fistulas in women.

A ureteral stent may be retained in the urinary tract for an extended period of time, such as several weeks or months. Ureteral catheters and stents alike may be positioned in the urinary tract using endoscopic techniques or percutaneously (stents only). The current standard procedure for the insertion of ureteral catheter uses a ureteroscope and X-Ray guidance and is performed on anesthetized patients. These conditions require the procedure to be performed in an operating room, thus increasing procedure cost and patient's morbidity.

Repeated insertion and removal of a catheter, in particular, indwelling catheter, such as ureteral catheters, entails repeated anesthesia and exposure to radiation of the patient, and increases the chances of damage to internal organs, such as uretrovesical valve (the orifice between the ureter and the bladder). Thus, reducing the number of such catheter insertions would dramatically reduce adverse effects for patients.

Various catheters have been disclosed. For example, U.S. Pat. No. 4,790,809 discloses ureteral stent. International Patent Application Publication No. WO 2013/134758 is directed to implanted tube and external interface for saline or drug delivery to paranasal sinuses. International Patent Application Publication No. WO 2015/198333 to inventors of the present invention, is directed to a connectable catheter.

Nevertheless, there is thus a need in the art for catheters that are connectable and that can specifically and securely connect within the subject body to form a continuous catheter system, that does not require the repeated insertion of indwelling catheters into the subject's body and which can further allow sustained release of substances into the target region while optionally heating the substances and/or target region.

### SUMMARY

According to some embodiments, there is provided a connectable catheter system, device and methods of using the same. In some embodiments, the catheter systems disclosed herein may be used for various applications for delivery or removal of various substances to various target body parts, organs or cavities. In some embodiments, the connectable catheter system and devices disclosed herein include an internal, indwelling part (stent), which has already been pre-inserted into a desired target location/organ within a subject's body, and a removable, external, intermediary part (catheter), which is capable of specifically and safely connect to the indwelling part, within the subject body, to form a continuous catheter system capable of transferring fluids or other substances from/to the external location to/from an internal organ/cavity, wherein the catheter can advantageously allow slow/extended release of substances of interest to the target organ and/or along its path, and optionally be used for heating the substances and/or the internal target region, without damaging neighboring, un-treated tissues. In some embodiments, the catheter system utilizes directional connection to form more than one separable, internal conduits.

As detailed herein below, the systems, devices and methods disclosed herein are advantageous over currently used methods and catheter systems, as they can be used to complement the operation of an indwelling stent or catheter and allow minimizing the repetition of insertion procedures of internal, indwelling stents to the target organ/cavity and thereby dramatically reducing risks involved therewith (such as, anesthesia, infections and physical damage to internal organs) as well as increasing safety of treatment and increasing the treatment efficiency, by allowing a slow/extended/sustained release administration of a substance of interest to target regions and/or by providing localized heating of target regions to improve treatment efficacy, without affecting un-treated tissues. By utilizing the system, devices and methods disclosed herein, the overall well-being of the treated subject is improved. In addition, the systems, devices and methods disclosed herein, where used for treating cancer, can reduce the chance of tumor cell seeding, if a tumor develops in said organ/cavity.

In some embodiments, the systems, devices and methods disclosed herein are advantageous, as they allow the specific, accurate, safe and reversible and optionally, directional connection of an indwelling stent that has been placed within the subject body (for example, at or in a target organ or cavity) and a main, intermediary catheter (which may be transiently inserted and removed from the subject body), wherein the connection is being made within the subject body, without the need for repeated insertions of the indwelling stent, which may be left in its internal location and reconnected at will to the intermediary catheter that may be transiently inserted as needed. In some embodiments, the connection that is being made within the subject body, does not require visual guidance.

In some embodiments, the systems, devices and methods disclosed herein are further advantageous, as they allow sustained/extended/slow release administration of substances of interest along the fluid conduit of the catheter, optionally into target organ, while maintaining safety and efficacy of treatment, without the need for repeated insertions of indwelling stent. In further embodiments, the systems, devices and methods disclosed herein are advantageous as they are capable of specifically and controllably provide heating of the substances of interest and/or the target tissue, without affecting or damaging in any way non-related, untreated neighboring tissues.

In some embodiments, the substance of interest is a medical substance. Is some embodiments, the substance is a therapeutic substance. In some embodiments, the substance is a drug. In some embodiments, the substance is an anticancer drug. In some embodiments, the substance is in a form of a gel. In some embodiments, the substance is in a form of a gel comprising one or more drugs. In some embodiments, the substance is a gel comprising mitomycin C.

According to some embodiments, there is thus provided a connectable catheter system comprising an intermediary catheter and an indwelling stent catheter. The intermediary catheter includes an external (proximal) section (end) and a tip (internal/distal) section (end) opposing thereto, the tip section is configured to be inserted into a body of a subject. The indwelling stent, configured to be located within the body of the subject, includes a connecting section (end) and a target section (end); wherein the connecting section of the indwelling stent is configured to reversibly connect, within the body of the subject, with the tip section of the intermediary catheter to form a continuous conduit (such as a continuous fluid passage) from the external section (end) of the intermediary catheter to the indwelling stent.

According to some embodiments, there is provided a connectable catheter system for providing extended release of substance in a target cavity, the system comprising: an intermediary catheter comprising an external section and a tip section, the tip section is configured to be inserted into a body of a subject; and a reconnectable indwelling stent comprising a connecting section and a target section, the target section being configured to be located within a body of the subject, wherein the indwelling stent comprises at lateral walls thereof, apertures configured to allow extended release of a substance administered therethrough; wherein the connecting section of the reconnectable indwelling stent is configured to reversibly connect, within the body of the subject, to the tip section of the intermediary catheter to form a continuous conduit between the intermediary catheter and the reconnectable indwelling stent.

In some embodiments, the apertures may traverse the outer walls of the indwelling stent to allow release of substances along the length of the indwelling stent. In some embodiments, the apertures may be identical, similar or different in shape, size and distribution along the length of the lateral walls of the indwelling stent. In some embodiments, the apertures may be capillary sized. In some embodiments, the apertures may be perpendicular relative to the flow of the substance in an internal conduit of the indwelling stent. In some embodiments, the apertures may be slanted at a negative angle relative to the flow of the substance in an internal conduit of the indwelling stent. According to some exemplary embodiments, the apertures may be bi-slanted.

According to some embodiments, there is provided a connectable catheter system for delivering a substance to a kidney of a subject, the system comprising: an intermediary catheter comprising an external section and a tip section, the tip section is configured to be inserted into a body of a subject; and a reconnectable indwelling stent comprising a connecting section and a target section, the target section being configured to be located within a kidney of a subject, wherein the indwelling stent comprises at the target section an anchoring member and an inflatable ureteropelvic junction (UPJ) balloon; wherein the connecting section of the reconnectable indwelling stent is configured to reversibly connect, within the body of the subject, to the tip section of the intermediary catheter to form a continuous conduit between the intermediary catheter and the reconnectable indwelling stent, and wherein the anchoring member is configured to anchor the indwelling stent within the kidney. In some embodiments, the anchoring member may be selected from a pig-tail anchoring member and a memory shape tip anchoring member.

In some embodiments, the indwelling stent may further include more than one internal conduits. In some embodiments, an internal conduit of the indwelling stent may be configured to provide fluid to inflate the UPJ balloon. In some embodiments, upon inflation of the UPJ balloon, blockade of the ureteropelvic junction is initiated/formed.

In some embodiments, the system may further include one or more heating elements configured to heat the substance and/or designated internal tissues.

According to some embodiments, there is provided a connectable catheter system for heating an internal target tissue of a subject, the system comprising: an intermediary catheter comprising an external section and a tip section, the tip section is configured to be inserted into a body of a subject; and a reconnectable indwelling stent comprising a connecting section and a target section, the target section being configured to be located within the body of the subject, at a target tissue, said indwelling stent comprises heating elements; wherein the connecting section of the reconnectable indwelling stent is configured to reversibly connect, within the body of the subject, to the tip section of the intermediary catheter to form a continuous conduit between the intermediary catheter and the reconnectable indwelling stent, and wherein the heating elements of the indwelling stent are configured to heat the internal target tissue of the subject.

In some embodiments, the indwelling stent may include more than one internal conduits. In some embodiments, directional connections between the intermediary catheter and the indwelling stent are formed utilizing diametrical magnets to form additional internal dedicated continuous conduits.

In some embodiments, the heating element may be selected from selected from a conductive element, an energy source, a fluid heating element and an infrared light emitting diode (LED). Each possilibty is a speprate embodiment. In some embodiments, the system may further include an external heat generating element capable of generating heat or energy. In some embodimetns, the external heat generating element is capable of (or configured to) transferring said heat or energy through a dedicated conduit to the heating element of the indwelling stent, to thereby allow the heating element to heat the target tissue. In some embodiments, the external heat generating element is configured to serve as an external heating source. In some embodiments, the heat or energy source may include, microwave heating, radiofrequency heating, electrical heating, chemical heating, infrared heating, ultrasound heating, fluid heating, or combinations thereof. Each possilibty is a separate embodiment. In some embodiments, the system may provide circulation of heat in the target cavity. In some embodiments, the heat may be conducted by a gel that is administered through an internal instillation lumen of the indwelling stent to the internal target tissue.

In some embodiments, the indwelling stent may further include an anchoring member, configured to anchor the target section of the indwelling stent in the target tissue. In some embodiments, the anchoring member may be selected from a pig-tail anchoring member and a memory shape tip anchoring member.

According to some embodiments, the substance is a therapeutic substance. In some embodiments, the substance is a viscous fluid in the form of a gel. In some embodiments, the gel comprises Mitomycin C (MMC).

According to some embodiments, there is provided a reconnectable indwelling stent that may include a target section configured to be located within a subject's body cavity, said target section comprises an anchoring member and one or more heating elements; a connecting section configured to reversibly connect, within the body, to a tip section of an intermediary catheter to form a continuous fluid conduit between the intermediary catheter and the reconnectable indwelling stent; and a main section located between said target section and said connecting section, wherein said connecting section is firmly attached to or integrally formed with the main section. In some embodiments, the reconnectable indwelling stent may include more than one internal dedicated conduits. In some embodiments, the reconnectable indwelling stent may further include at lateral external walls thereof, apertures, allowing controlled substance passage from the interior of the indwelling stent to the exterior.

According to some embodiments, there is provided a connectable catheter system, comprising: an intermediary catheter comprising an external section and a tip section, the tip section is configured to be inserted into a body of a subject; and a reconnectable indwelling stent comprising a connecting section and a target section, the target section being configured to be located within a body of the subject, wherein the connecting section of the reconnectable indwelling stent is configured to reversibly connect, within the body, to the tip section of the intermediary catheter to form a continuous fluid conduit between the intermediary catheter and the reconnectable indwelling stent.

According to some embodiments, the connecting section of the reconnectable indwelling stent is configured to fit into an inner lumen of the tip section of the intermediary catheter.

In some embodiments, the inner lumen of the intermediary catheter may include a securing member configured to secure the connecting section of the reconnectable indwelling stent within the inner lumen of the tip section of the intermediary catheter. In some embodiments, the securing member may include a deployable securing member, vacuum ducts, a flexible plate, matching recesses in the connecting section of the reconnectable indwelling stent and the inner lumen of the tip section of the intermediary catheter, or any combination thereof. In some exemplary embodiments, the securing member may include one or more inflatable balloons, configured to be inflated within the inner lumen of the intermediary catheter. In some embodiments, the intermediary catheter may further include one or more inflating port(s) configured to inflate the one or more inflatable balloons.

In some embodiments, the securing member may be further configured to seal a fluid passage between the outer wall of the reconnectable indwelling stent and the inner wall of the intermediary catheter.

According to some embodiments, the intermediary catheter may further include a sealing member configured to seal a fluid passage between the outer wall of the reconnectable indwelling stent and the inner wall of the intermediary catheter. In some embodiments, the sealing member is configured to prevent fluid leakage between the intermediary catheter and indwelling stent. In some embodiments, the sealing member may prevent or overcome decoupling forces that may be exerted when the fluid provided is viscous.

In some embodiments, the system may further include a stylet removably insertable into the inner lumen of the intermediary catheter, the stylet includes, at a tip section thereof, an attachment member configured to capture and/or attach to the connecting section of the reconnectable indwelling stent, such that when the stylet is proximally retracted within the inner lumen of the intermediary catheter, the reconnectable indwelling stent is pulled into the inner lumen of the intermediary catheter. In some embodiments, the stylet's attachment member may include a magnet configured to attract the connecting section of the reconnectable indwelling stent. In some exemplary embodiments, the stylet's attachment member may include a magnet configured to attract a corresponding magnet that is located on the connecting section of the indwelling stent, and which is integrally formed therewith.

According to some embodiments, the connecting section of the indwelling stent may include a magnet or a component attractable by the stylet's magnet. That magnet or component attractable by the stylet's magnet may be configured to be placed or formed around the indwelling catheter lumen such that passage of fluids therethrough is enabled.

In some embodiments, the connecting section of the reconnectable indwelling stent may include a magnet or a component attractable by the stylet's magnet.

In some embodiments, the stylet's attachment member may include a loop, lasso, protrusion, pigtail, net, basket structure or any combination thereof. In some embodiments, the connecting section of the indwelling stent is fitted with a corresponding connection member that may be an integrated part of the stent luminal tube. In some embodiments, the connecting section of the reconnectable indwelling stent may include a loop, protrusion, lasso, pigtail or any combination thereof.

In some embodiments, the stylet may further include or be functionally associated with an indication unit configured to provide an indication of a connection between the attachment member of the stylet and the connecting section of the reconnectable indwelling stent.

In some embodiments, the reconnectable indwelling stent may include a stopper element at an outer wall thereof, wherein the stopper element may be located between the connecting section and the target section and wherein the stopper element is configured to limit the length (portion) of the reconnectable indwelling stent that can enter the inner lumen of the intermediary catheter.

In some embodiments, the system may further include a guide rod removably insertable into the inner lumen of the connectable catheter, the guide rod is configured to facilitate the penetration of the tip section of the intermediary catheter into the subject's body. In some embodiments, the guide rod is comprised with an a-traumatic tip and/or a flexible end-section.

In some embodiments, the guide rod may be further configured to remove the reconnectable indwelling stent from the intermediary catheter when pushed distally within the inner lumen of the connectable catheter.

In some embodiments, the intermediary catheter may further include a deployable anchoring element located at an outer wall thereof, the deployable anchoring element, when deployed, is configured to anchor the intermediary catheter in the subject's body.

In some embodiments, the intermediary catheter may further include opening(s) or administration port(s) for allowing the transfer of fluids and/or various devices (such as, guide-wires). In some embodiments, the intermediary catheter may include a one or more connector(s) and/or one or more administration port(s), such as, a medical substance administration port.

In some embodiments, the system may further include a control circuitry unit configured to provide an indication when the continuous fluid conduit is formed.

In some embodiments, the system may further include a vacuum source configured to create vacuum in the inner lumen of the intermediary catheter to secure the positioning of the reconnectable indwelling stent.

According to some embodiments, there is provided an intermediary catheter, comprising: an external section and a tip section, the tip section is configured to be inserted into a body of a subject; a securing member located at an inner lumen of the intermediary catheter, the securing member is configured to secure a connecting section of an reconnectable indwelling stent within the inner lumen of the tip section thereof and thereby to form a continuous fluid conduit between the inner lumen of the intermediary catheter and an inner lumen of the reconnectable indwelling stent.

In some embodiments, the securing member may include a deployable securing member, vacuum ducts, matching recesses in the connecting section of the reconnectable indwelling stent and the inner lumen of the tip section of the intermediary catheter, or combinations thereof. In some embodiments, the securing member comprises one or more inflatable balloons.

In some embodiments, the intermediary catheter may further include an inflating port configured to inflate the one or more inflatable balloons.

In some embodiments, the securing member may be further configured to seal a fluid passage between an outer wall of the reconnectable indwelling stent and the inner wall of the intermediary catheter. In some embodiments, the intermediary catheter may further include a sealing member configured to seal a fluid passage between the outer wall of the reconnectable indwelling stent and the inner wall of the intermediary catheter. In some embodiments, the intermediary catheter may further include a deployable anchoring element located at an outer wall thereof, the deployable anchoring element, when deployed, is configured to anchor the intermediary catheter in the subject's body. In some embodiments, the intermediary catheter may further include a one or more connectors(s) and/or one or more administration port(s), such as, a medical substance administration port.

In some embodiments, the intermediary catheter may be functionally associated with a control circuitry unit configured to provide an indication when the continuous fluid conduit is formed.

In some embodiments, the intermediary catheter may be functionally associated with a vacuum member configured to create vacuum in the inner lumen of the intermediary catheter to secure the positioning of the reconnectable indwelling stent.

According to some embodiments, there is provided a reconnectable indwelling stent comprising: a target section configured to be located within a subject's body cavity; a connecting section configured to reversibly connect, within the body, to a tip section of an intermediary catheter to form a continuous fluid conduit between the intermediary catheter and the reconnectable indwelling stent; and a main section located between said target section and said connecting section, wherein said connecting section is firmly attached to or integrally formed with the main section.

In some embodiments, the connecting section may include a magnet or a component attractable by a magnet. In some embodiments, the connecting section may include a loop, lasso, pigtail or any combination thereof.

In some embodiments, the reconnectable indwelling stent may further include a stopper element at an outer wall thereof, wherein the stopper element is located on the main section and wherein the stopper element is configured to limit the portion (such as length) of the reconnectable indwelling stent that can enter the inner lumen of the intermediary catheter.

In some embodiments, the reconnectable indwelling stent may further include one or more drainage holes at a wall thereof.

In some embodiments, the reconnectable indwelling stent may be configured to transfer fluids from the body cavity and/or to the body cavity after formation of the continuous fluid conduit.

According to some embodiments, there is provided a stylet for removable insertion into an inner lumen of an intermediary catheter, the stylet comprises, at a tip section thereof, an attachment member configured to attach, within a subject's body, to the connecting section of a reconnectable indwelling stent, such that when the stylet is proximally retracted within the inner lumen of the intermediary catheter, the reconnectable indwelling stent is pulled into the inner lumen of the intermediary catheter.

In some embodiments, the attachment member of the stylet may include a magnet or a component attractable by a magnet configured to attract the connecting section of the reconnectable indwelling stent.

In some embodiments, the stylet's tip section is flexible. In some embodiments, the stylet's tip section may include a shape memory material configured to obtain a bent configuration upon exiting a tip section of the intermediary catheter.

In some embodiments, the stylet's attachment member may include a loop, a lasso, a pigtail, a net, a basket structure, or any combination thereof.

According to some embodiments, there is provided a method of connecting a connectable catheter system, the method comprising: drawing a connecting section of a reconnectable indwelling stent into an inner lumen of the intermediary catheter; securing the connecting section of the reconnectable indwelling stent within the inner lumen of a tip section of the intermediary catheter, thereby forming a continuous fluid conduit between the inner lumen of the intermediary catheter and an inner lumen of the reconnectable indwelling stent; and utilizing a control circuitry unit, providing an indication when the continuous fluid conduit is formed.

In some embodiments, securing the connecting section of the reconnectable indwelling stent within the inner lumen of the tip section of the intermediary catheter may include deploying a securing member located at an inner lumen of the intermediary catheter.

In some embodiments, drawing (pulling) a connecting section of the reconnectable indwelling stent into the inner lumen of the intermediary catheter may be conducted by inserting a stylet into the inner lumen of the intermediary catheter, the stylet comprises, at a tip section thereof, an attachment member configured to attach to the connecting section of the reconnectable indwelling stent, such that when the stylet is proximally retracted within the inner lumen of the intermediary catheter, the reconnectable indwelling stent is pulled into the inner lumen of the intermediary catheter.

In some embodiments, the method may further include automatically providing, utilizing an indication unit, an indication when a connection between the attachment member of the stylet and the connecting section of the reconnectable indwelling stent is formed.

According to some embodiments, there is provided a method of treating cancer, the method comprising administering an anti-cancer drug to a subject in a need thereof, utilizing the connectable catheter system disclosed herein. In some embodiments, the anti-cancer drug is administered to a target organ/cavity. In some embodiments, the cancer is a urinary tract cancer.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIGS. 1A-B—Schematic perspective views of parts of a connectable catheter system, according to some embodiments;

FIG. 2—a schematic perspective view of an intermediary catheter, according to some embodiments;

FIGS. 3A-B—schematic perspective illustrations of a connectable catheter system located within an internal body region, according to some embodiments;

FIGS. 4A-C—schematic perspective illustrations of an exemplary connection establishment, within a subject body between the intermediary catheter and the indwelling stent, according to some embodiments; FIG. 4A—prior to connection; FIG. 4B—after connection; FIG. 4C—after connection and insertion of the indwelling stent into the intermediary catheter;

FIGS. 5A-D—schematic close-up views of a cross section of the tip section of an intermediary catheter being connected to an indwelling stent, according to some embodiments; FIG. 5A—prior to removal of stylet; FIG. 5B- after removal of stylet and formation of a continuous fluid conduit; FIG. 5C—prior to removal of stylet and prior to securing and/or sealing the connection; FIG. 5D—after removal of stylet and formation of a secured and sealed continuous fluid conduit between the indwelling stent and the intermediary catheter;

FIGS. 6A-D—schematic illustrations of exemplary means for approximating and/or connecting and/or securing the connection of the tip section of an intermediary catheter with the connecting section of the indwelling stent, according to some embodiments; FIGS. 6A-6B—connection with magnets; FIG. 6C—connection using a three dimensional basket structure; FIG. 6D—attachment and securing of the connection using vacuum ducts;

FIGS. 7A-C—schematic illustrations of exemplary diametrical magnets that can be used for establishing a directional connection between intermediary catheter and indwelling stent, according to some embodiments.

FIGS. 8A-E—schematic illustrations of exemplary means of forming directional connection of an intermediary catheter with indwelling stent using diametrical magnets, according to some embodiments. FIG. 8A shows a top view of the connecting tip of an intermediate catheter; FIG. 8B shows a side view of the connecting tip of an intermediate catheter. FIG. 8C shows a bottom view of the tip of the intermediate catheter. FIG. 8D and FIG. 8E—show schematic views of exemplary magnets having more than one magnetic conduits.

FIG. 9—schematic illustration of an exemplary radial connection, allowing an electrical connection in the connectable catheter system, according to some embodiments.

FIGS. 10A-B—schematic illustrations of exemplary direct stylet to stent connection utilizing magnets, according to some embodiments;

FIGS. 11A-11B—schematic illustrations of intermediate catheter system utilizing inflatable balloons at a tip thereof, according to some embodiments; FIG. 11A—the balloons are at an inflated state; FIG. 11B—The balloons are at their inflated state.

FIGS. 12A-12D—schematic illustrations of a cross section of external lateral wall of an indwelling stent having apertures/holes in its lateral sides, according to some embodiments;

FIG. 13—a schematic illustration of anchoring members of indwelling catheter, according to some embodiments;

FIG. 14—a schematic illustration of anchoring members of indwelling catheter, according to some embodiments;

FIGS. 15A-C—schematic illustrations of exemplary means for providing heat to a target cavity, according to some embodiments;

FIGS. 16A-16B—schematic illustrations of a dual lumen indwelling catheter with a balloon for ureteropelvic junction (UPJ) for urine blockage.

FIG. 17—a flow chart diagram showing steps in a method of connecting a connectable catheter system and optionllay providing heat to a target cavity, according to some embodiments; and

FIGS. 18A-18B—illustration of an exemplary use of the connectable catheter system after positioning in a urinary tract prior to (FIG. 18A) or after (FIG. 18B) attachment of the indwelling stent to the intermediary catheter.

### DETAILED DESCRIPTION

According to some embodiments, there is provided an advantageous connectable catheter system and devices, configured to being connected within a body of a subject to form a continuous catheter, extending from outside the body to an internal target region of the body, such as an organ, a cavity, and the like. The connectable catheter system, device and method disclosed herein provide a cost effective, safe, robust and efficient catheter system allowing the prolonged use of an indwelling stent placed within a subject's body (for example, in a target organ or cavity). In some embodiments, by utilizing a connectable catheter system, it is possible to avoid repeated insertion of the stent part (member) for each administration of therapy. For example, this is particularly advantageous when the insertion into the target cavity may pose hurdles, such as, it may cause damage, may require special means (such as medical imaging) and/or or special clinician skills. By utilizing the systems and methods of the current disclosure, the stent is inserted/placed only once for a defined set of treatments and only the intermediary catheter needs to be inserted for each treatment session. Thus, the disclosed systems and methods are advantageous as they allow a simpler, cost effective, safe and efficient which can induce less damage to the treated internal cavity or tissue, and increases overall treatment efficiency and patient compliance.

### Definitions

To facilitate an understanding of the present invention, a number of terms and phrases are defined below. It is to be understood that these terms and phrases are for the purpose of description and not of limiting, such that the terminology or phraseology of the present specification is to be interpreted by the skilled artisan in light of the teachings and guidance presented herein, in combination with the knowledge of one of ordinary skill in the art.

As used herein, the term “catheter” is directed to a tube having a continuous, internal lumen and two opposing ends/sections. The two ends may include an external end (located externally to a body of a subject) and an internal end (capable of being located within a body of the subject, for example, in an organ or within a cavity). In some instances, the catheter is configured to allow the transfer of various materials (for example, fluids (including gaseous fluids or liquids)) between (to and from) regions external and internal to the body. For example, the catheter may be used to transfer liquids (such as urine) from the bladder (internal organ) to an external location (such as external collecting bag). In some embodiments, the catheter may be rigid, semi-rigid or flexible and may be made of various materials. In some embodiments, the catheter may have various dimensions (length, external diameter, internal diameter, and the like). In some embodiments of the present invention, the catheter may be comprised of at least two parts/portions that may connect within the body of the subject to form a continuous catheter tube extending from outside the body of a subject, to an internal location (such as an organ or a cavity). Exemplary internal organs or cavities into which a catheter may be inserted include such organs as, but not limited to: urinary tract (renal pelvis, bladder), heart, intestinal system, uterus, lungs, pleura, gastrointestinal tract, nasal sinus, esophagus, rectum, vagina, stomach, abdomen, peritoneum, liver, kidney and brain, and the like.

As use herein, the terms “main catheter”, “connecting catheter” and “intermediary catheter” may be used interchangeably.

As used herein, the term “indwelling” is directed to an article (a device) which is inserted to and located within a subject body for an extended period of time (for example, between several hours and until 12 month; between 12 hours to 8 months, between 1 day to 6 months, between 1 week to 4 months, between 2 weeks to 3 months, between 4 weeks to 2 months). As used herein, the terms “indwelling device”' “indwelling stent”, “indwelling catheter” “reconnectable indwelling catheter”, “reconnectable indwelling stent”, and “reconnectable indwelling device” may be used interchangeably.

As used herein, the term “proximal” is directed to a location which is external to a subject's body and close to the clinician. As used herein, the term “distal” is directed to a location which is internal to a subject body and further away from the clinician.

As used herein, the terms “target organ”, “target tissue”, “target cavity”, “internal target organ”, “internal target cavity” may be used interchangeably and are directed to an internal organ/cavity into which or from which materials may be transferred from/to an external location. In some embodiments, the target section of the indwelling stent is located within the target organ.

As used herein, the terms “cavity” and “body lumen” may interchangeably be used. The terms are directed to a closed compartment/structure/organ/tissue, within a subject body. Exemplary cavities may include such cavities as, but not limited to: stomas, bladder, kidney, heart, intestines, uterus, lungs, urinary tract, and the like.

According to some embodiments, the present invention provides a connectable catheter system, the catheter system includes a first, (at least partially external) intermediary (main) catheter and a second, internal indwelling stent, whereby the intermediary catheter and the indwelling stent may be connected, internally, within a subject body to form a continuous conduit (passage), capable of transferring materials (such as fluids) to and from an external location (where one end of the intermediary catheter is located) to an internal body location (such as an organ or cavity), where an indwelling stent is located. In some embodiments, the system can further allow direct or indirect heating of target regions, to enhance treatment efficiency. In some embodiments, the system can further allow extended release administration of therapeutic substances along the entire path of the catheter or along a portion of said path.

According to some embodiments, the indwelling stent is configured to be inserted into an internal body location (such as an organ/cavity). In some embodiments, the indwelling stent is configured to be retained in the body, for an extended period of time, so as to allow and enable the performance of a number of uses, without the need to repeatedly remove it from the body. According to some embodiments, the target section of the indwelling stent may be fitted with a reversible fixing (anchoring) element that may assist in retaining the indwelling stent in the target area/cavity. For example, when used in the urinary tract, the target section of an indwelling urinary stent may be kept in the renal pelvis by a looped end such that the fixing element may be a string that locks the looped end or a nitinol made member that can ruggedize the looped end to thereby fix the indwelling stent.

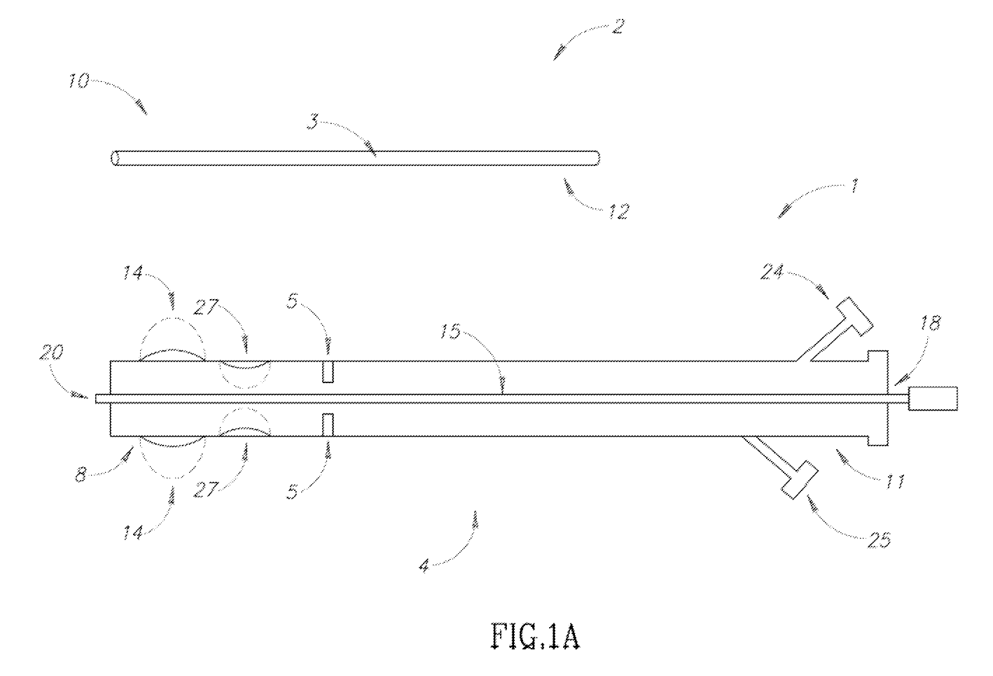

Reference is now made to FIG. 1A, which is a schematic illustration of a catheter system, according to some embodiments. As shown in FIG. 1A, the connectable catheter system (1) is comprised of at least two connectable parts/portions: an indwelling stent part (2) and an intermediary catheter part (4). The indwelling stent (2), shown in cross-section in FIG. 1A, is illustrated in the form of a tube (optionally flexible), having an internal lumen (3). Indwelling stent (2) has two opposing ends/sections: a “connecting section” (12) and a “target section” (10), such that target section (1) is closer to (or within) an internal target organ/cavity. In some embodiments, connecting section (12) of indwelling stent (2) may be positioned in or adjacent to an internal body location, and target section (10) may be positioned inside or in close proximity to the target organ/cavity (for example, but not limited to, the renal pelvis).

As further shown in FIG. 1A, intermediary (main) catheter (4) (cross-section depiction) includes two opposing sections (ends): an external (proximal) section (11), and a tip (distal/internal) section (8). As further detailed below, external section (11) includes opening(s) and/or connector(s) that are required for the catheter operation. Tip section (8) is configured to be inserted to a body of a subject.

As further shown in FIG. 1A, intermediary catheter (4) includes, in an internal longitudinal lumen thereof, a stylet (15), between proximal and tip sections (11 and 8, respectively) insertable through opening (18). A tip section (20) of stylet (15), when fully inserted through the internal longitudinal lumen of intermediary catheter (4) protrudes from tip section (8) of intermediary catheter (4). Tip section (20) of stylet (15) is configured to connect to connecting section (12) of indwelling stent (2) and pull it into the inner lumen of intermediary catheter (4). Thereby, when stylet (15) is pulled out from intermediary catheter (4), a continuous fluid conduit is formed between internal lumen (3) of indwelling stent (2) and the internal lumen of intermediary catheter (4). Barrier (5), located in the internal longitudinal lumen of intermediary catheter (4), is configured to limit the retraction of stylet (15), when connected to indwelling stent (2). Intermediary catheter (4) further includes an inflation conduit (not shown), extending from inflation port (24) to an inflatable anchoring balloon (14) located at an external surface of tip section (8) of intermediary catheter (4). Inflation of inflatable anchoring balloon (14) may be achieved by connecting a source of fluid (such as, gas (for example, air), or liquid (such as, water, sterile saline)), to inflation port (24) located at the external section of intermediary catheter (4), wherein the inflation may be conducted utilizing a valve, such as a check valve. Inflatable anchoring balloon (14) is represented in an inflated configuration by a dashed line and in a deflated configuration by a solid line. Intermediary catheter (4) further includes, at an inner lumen thereof, a securing member (27). Securing member (27) is configured to secure connecting section (12) of indwelling stent (2) within the inner lumen of an intermediary catheter (4), in tip section (8) thereof. Securing member (27) is represented in an inflated configuration by a dashed line and in a deflated configuration by a solid line. Intermediary catheter (4) further includes an additional inflation conduit (not shown), extending from inflation port (25) to securing member (27). Securing member (27) may further be configured to seal fluid passage outside of indwelling stent (2). In some embodiments, as further detailed below, the intermediary catheter includes a separate sealing member in addition to the securing member.

Reference is now made to FIG. 1B, which is a schematic illustration of a catheter system, according to some embodiments. As shown in FIG. 1B, the connectable catheter system (50) is comprised of at least two connectable parts/portions: an indwelling stent part (52) and an intermediary catheter part (54). The indwelling stent (52), shown in cross-section in FIG. 1B, is illustrated in the form of a tube (optionally flexible), having an internal lumen (53). Indwelling stent (52) has two opposing ends/sections: a “connecting section” (62) and a “target section” (60). In some embodiments, connecting section (62) of indwelling stent (52) may be positioned in or adjacent to an internal body location, and target section (60) may be positioned inside or in close proximity to the target organ/cavity (for example, but not limited to, the renal pelvis).

As further shown in FIG. 1B, intermediary (main) catheter (54) (cross-section depiction) includes two opposing sections (ends): an external (proximal) section (61), and a tip (distal/internal) section (58). As further detailed below, external section (61) includes opening(s) and/or connector(s) that are required for the catheter operation. Tip section (58) is configured to be inserted to a body of a subject.

As further shown in FIG. 1B, intermediary catheter (54) includes, in an internal longitudinal lumen thereof, an internal catheter (56), insertable between proximal and tip sections (61 and 58, respectively) through opening (78). A tip section (70) of internal catheter (56), when fully inserted through the internal longitudinal lumen of intermediary catheter (54) protrudes from tip section (58) of intermediary catheter (54). Tip section (70) of internal catheter (56) is configured to connect to connecting section (62) of indwelling stent (52) and form a continuous fluid conduit. Intermediary catheter (54) further includes an inflation conduit (not shown), extending from inflation port (72) to an inflatable anchoring balloon (64) located at an external surface of tip section (58) of intermediary catheter (54). Inflation of inflatable anchoring balloon (64) may be achieved by connecting a source of fluid (such as, gas (for example, air), or liquid (such as, water, sterile saline)), to inflation port (72) located at the external section of intermediary catheter (54), wherein the inflation may be conducted utilizing a valve, such as a check valve. Inflatable anchoring balloon (64) is represented in an inflated configuration by a dashed line and in a deflated configuration by a solid line.

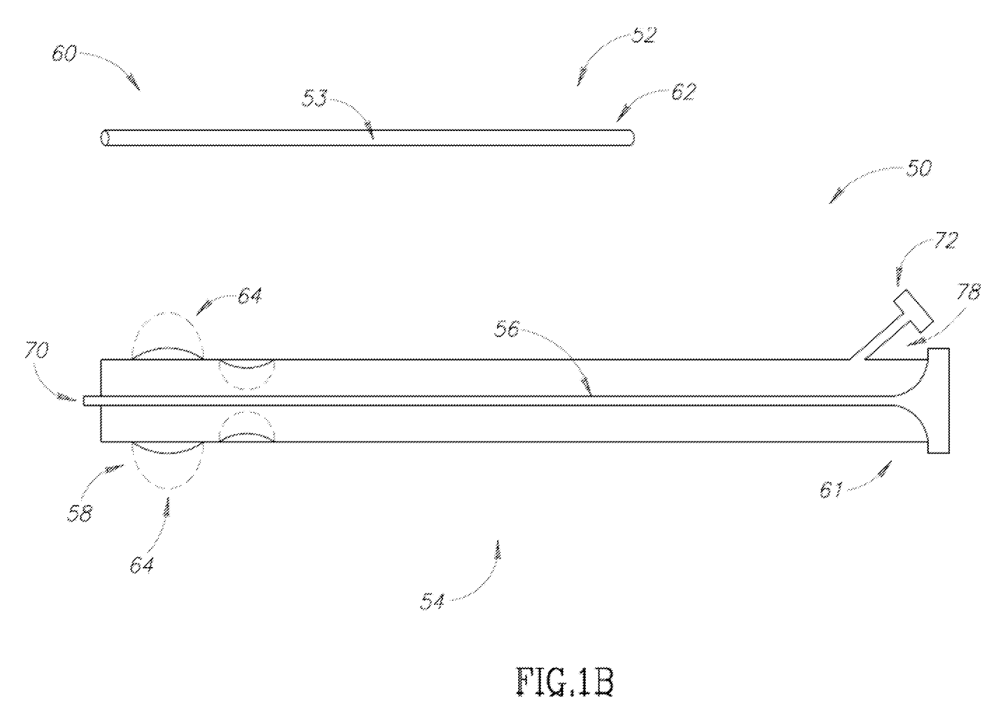

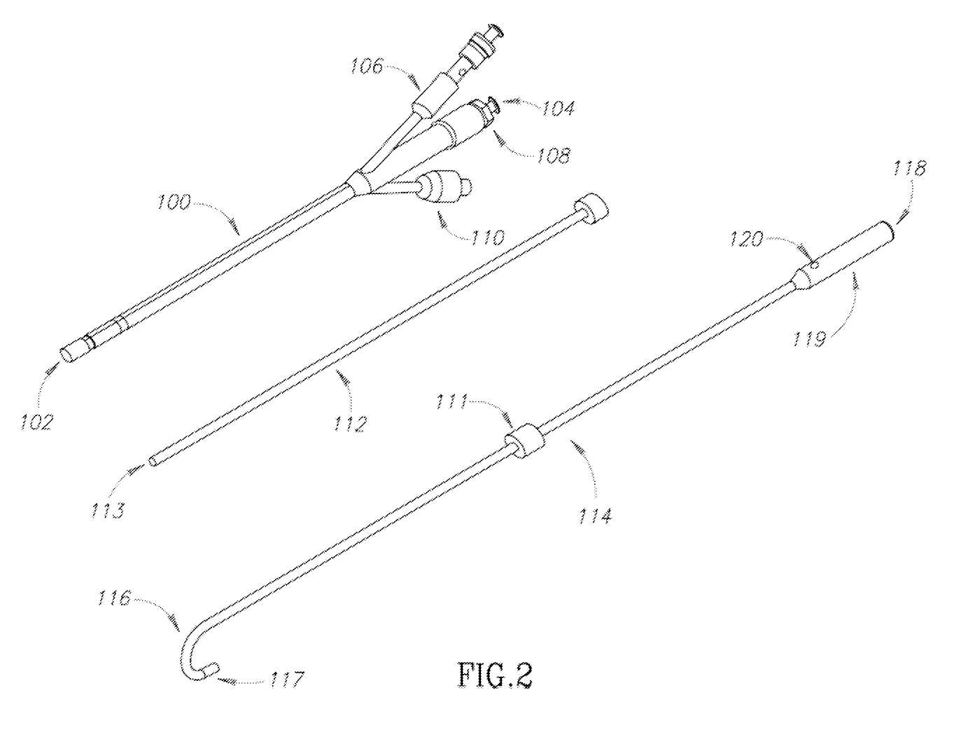

Reference is now made to FIG. 2, which is a schematic illustration of an intermediary (main) catheter, according to some embodiments. As shown in FIG. 2, intermediary catheter (100) has an external (proximal) section (104) and a tip (distal/internal) section (102). Intermediary catheter (100) has one or more openings or connectors or ports at the external section thereof, each configured to be connected to one or more devices and/or transfer different materials, via one or more inner lumens, that may be parallel to each other and may be identical, similar or different in length, composition or diameter. The exemplary intermediary catheter (100) illustrated in FIG. 2, includes three connectors (shown as connectors 106, 108 and 110). For example, connector 106 may be used to connect to a fluid transferring device (such as a syringe), for transferring fluids (for example, via an internal lumen located within the walls of the intermediary catheter), that can, for example, inflate/deflate an internal securing element, such as a securing balloon, or a sealing member, located on the inner walls of the intermediary catheter, the balloon is configured to secure the indwelling stent within the intermediary catheter, as further detailed below. For example, connector 110 may be used to connect to a fluid transferring device (such as syringe), for transferring fluids that can, for example, inflate/deflate an anchoring balloon, located on (or associated with) the outer walls of intermediary catheter (100), configured to secure the intermediary catheter in an internal body location (such as a body organ or cavity), as further detailed below. For example, connector 108 may be used to connect to a fluid transferring device (such as syringe), for transferring fluids from external section (104) of intermediary catheter (100) into the subject body. For example, connector (108) may be used to connect/guide various articles/devices that may be used in the placement and/or operation of the intermediary catheter. For example, guide rod (112) may be inserted via connector (108), through an inner lumen of intermediary catheter (100), such that that tip (113) thereof may protrude through tip section (102) of intermediary catheter (100) and may be used for guiding the insertion of the intermediary catheter into the subject body. In some embodiments, the guide rod may be a guide wire or mandrel. In some embodiments, as further detailed below, guide rod (112) may further be used for removing/disconnecting/pushing the indwelling stent from the intermediary catheter. For example, a stylet (shown as stylet 114) may be inserted via connector 108, through the inner lumen of intermediary catheter (100) such that it may protrude through the tip section (102) thereof and may be used for catching, connecting and/or maneuvering the indwelling stent. As shown in FIG. 2, exemplary stylet (114) has a tip section (tip 116) which includes at the tip a connecting member (117), that may specifically interact and connect with an internal indwelling stent. The external section of stylet (118) may include a handle (119) that may be used for controlling and maneuvering stylet (118). Stylet (114) may further include a connector (111) allowing the connection and/or securing of the stylet to intermediary catheter (100). The external section of stylet (118) may further include or associate with various indicators, such as a connection indicator (120) which is configured to provide an indication, such as: that a connection between the intermediary catheter (for example, via the stylet) and the indwelling stent has been achieved (as further detailed below); an indication that a continuous fluid passage (conduit) is formed between the inner lumens of the intermediary catheter and the indwelling catheter, and the like. In some embodiments, the stylet may further include one or more internal functional conduits. The intermediary catheter may further include a securing member located at an inner lumen of the tip section thereof, wherein the securing member is configured to secure a connecting section of an indwelling stent within the inner lumen of the tip section of the intermediary catheter, to allow formation of a continuous fluid conduit between the inner lumen of the connectable catheter and an inner lumen of the indwelling stent. The securing member may further be used as a sealing member, configured to seal the continuous fluid conduit between the inner lumen of the connectable catheter and an inner lumen of the indwelling stent. In some embodiments, a separate sealing member is used to seal the continuous fluid conduit between the inner lumen of the connectable catheter and an inner lumen of the indwelling stent. In some embodiments, the sealing member can prevent or overcome decoupling forces that may be exterted when the fluid provided is viscous

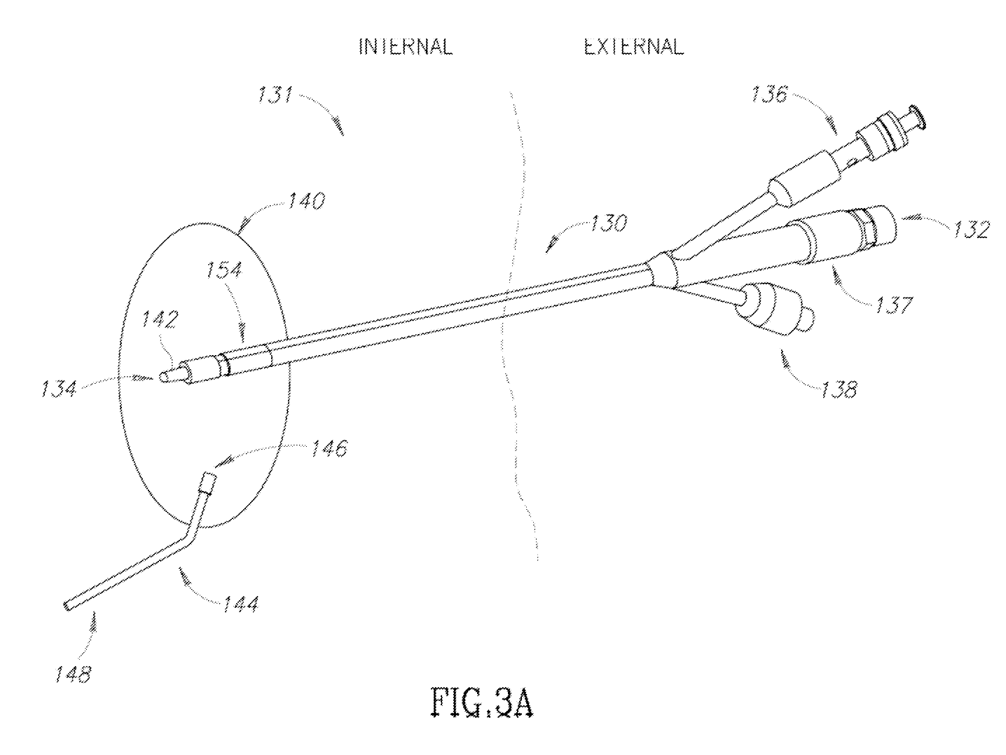

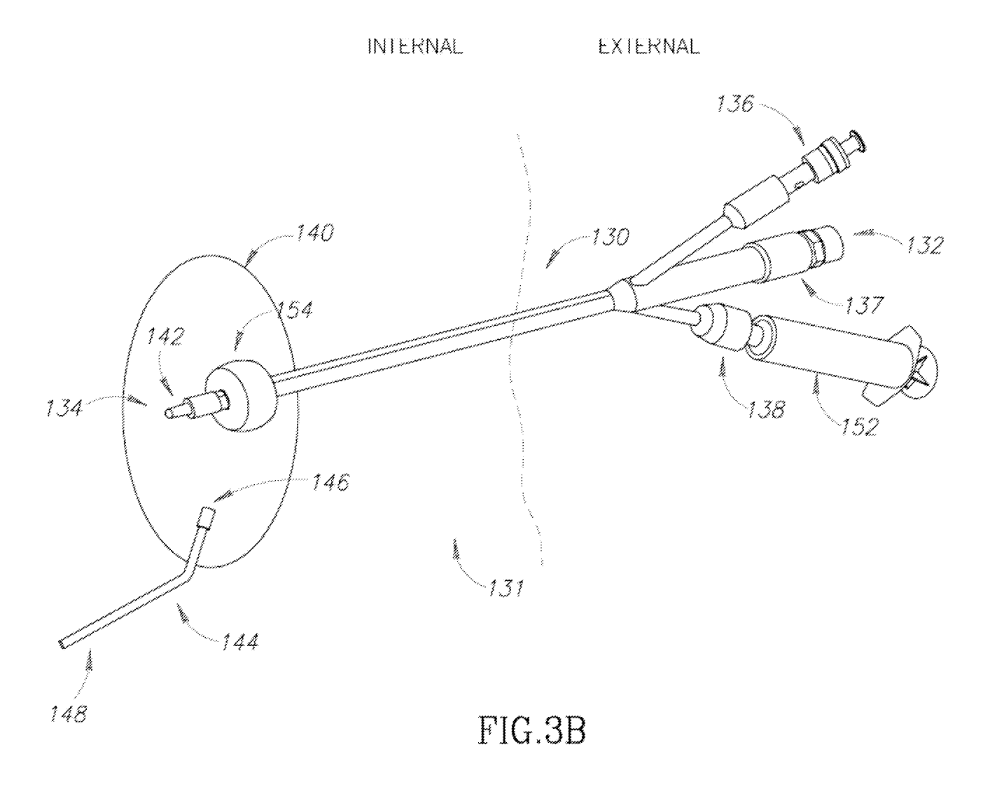

Reference is now made to FIGS. 3A-B, which are schematic perspective illustrations of components of a connectable catheter system (131) located within an internal body region, according to some exemplary embodiments. As shown in FIG. 3A, intermediary catheter (130) includes three connectors (136, 137 and 138) at the external section (132) thereof, which is located outside the body (“External”). The tip section (134) of intermediary catheter (130) is located within the body (“Internal”), for example, within a body cavity (140). The dashed line (131) is an imaginary line, separating between external body region (“External”) and internal body region (“Internal”). In the exemplary system shown in FIG. 3A, protruding through the inner lumen and from the tip section (134) of intermediary catheter (130), a distal tip (142) of a guide rod is shown. Anchoring mechanism (154), shown in the form of a deflated balloon, is located on an external surface of intermediary catheter (130). Further shown in FIG. 3A is indwelling stent (144), having its connecting section (146) located within body cavity (140) and the target section (148) located, for example, within a target organ/cavity. As shown in FIG. 3B, a syringe (152) is connected to connector (138), wherein the syringe is used to activate the anchoring mechanism by inflating balloon (154). The anchoring mechanism/element (such as anchoring balloon 154) is used to lock/fix the intermediary catheter in its internal location within the subject body (such as, within a body cavity).

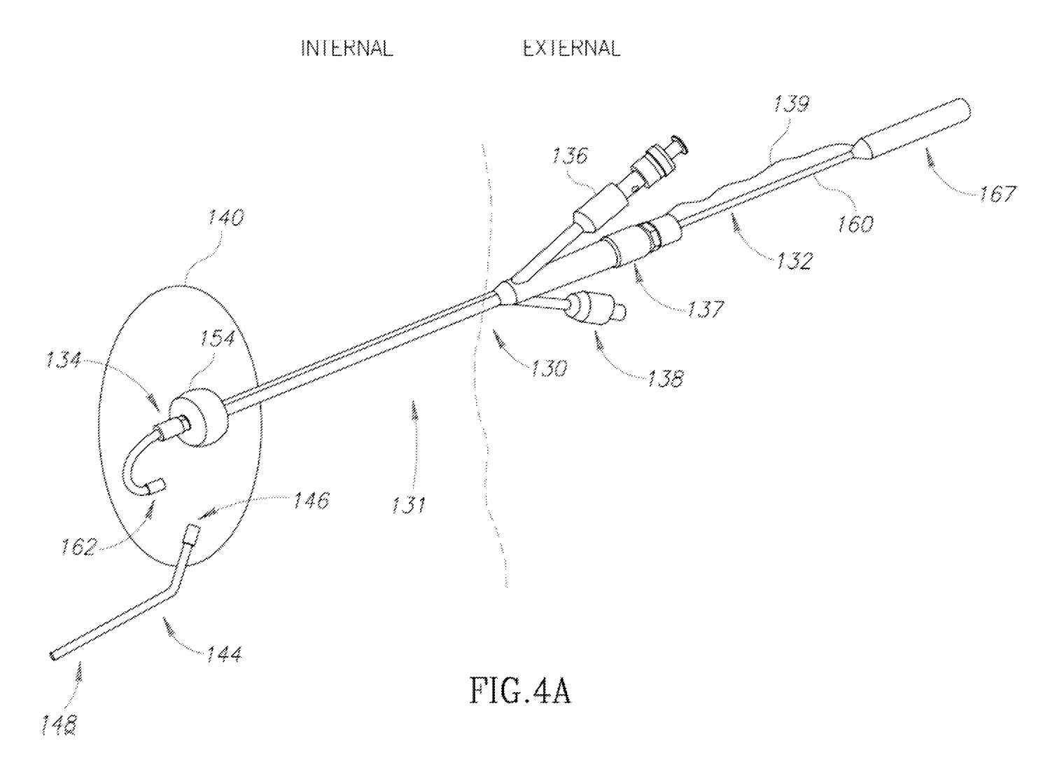

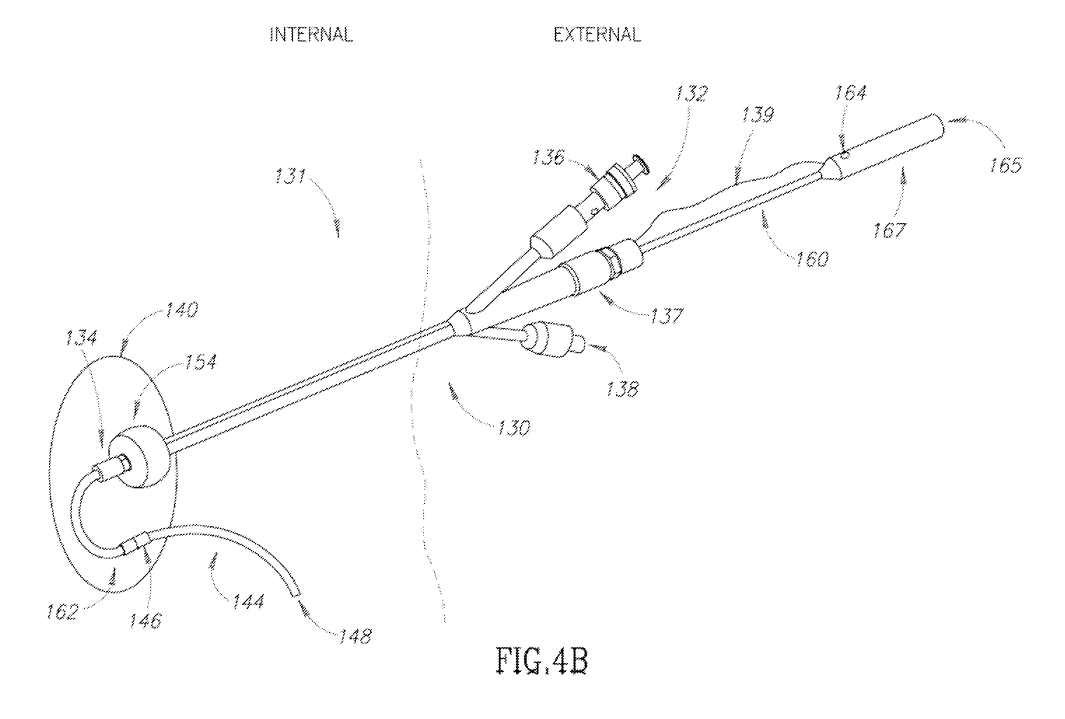

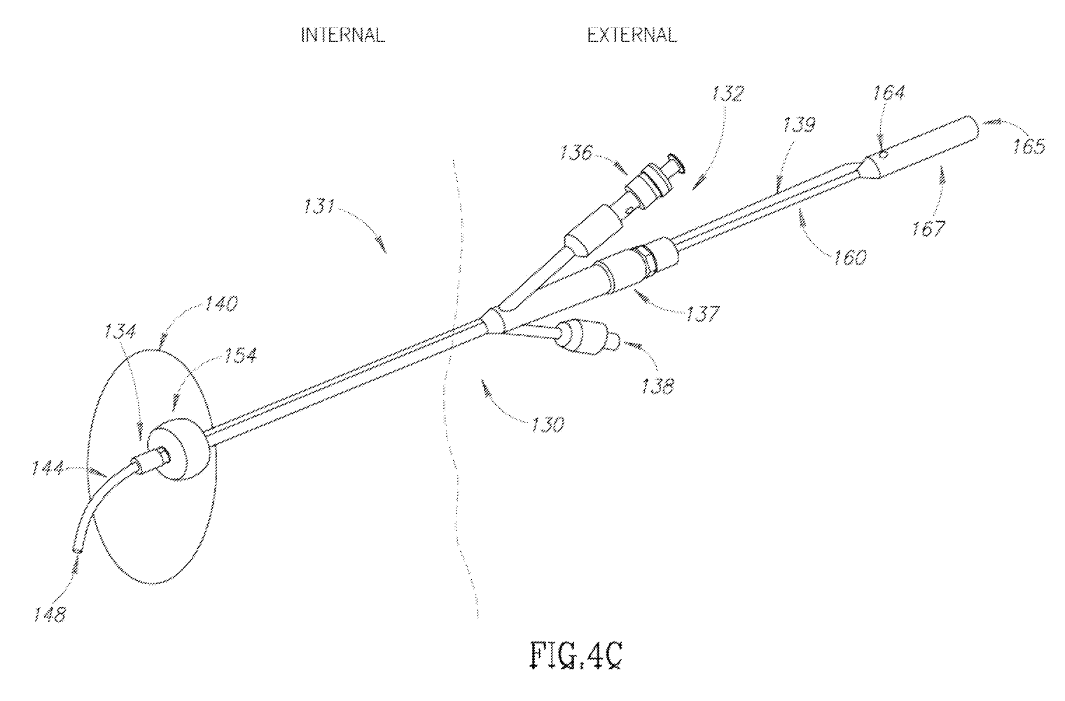

Reference is now made to FIGS. 4A-C, which are schematic perspective illustrations of exemplary connection establishment, within a subject body between the intermediary catheter and the indwelling stent, according to some embodiments. As shown in FIG. 4A, to exemplary system (131) presented in FIGS. 3A-B, an exemplary stylet (160), may be removably inserted from external section (132) of intermediary catheter (130), for example, via, connector (137), through the inner lumen of intermediary catheter (130) and protruding through tip section (134) of intermediary catheter (130). The tip section (162) of stylet (160) may be associated with or integrally formed with an attachment member configured to attach/connect to connecting section (146) of indwelling stent (144). Handle (167) of stylet (160) may be used to operate and/or maneuver the stylet. Further shown is string (139), in a relaxed position. As shown in FIG. 4B, tip section (162) of stylet (160) is connected to connecting section (146) of indwelling stent (144). The connection between the tip section of the stylet and the connecting section of the indwelling stent may be achieved by various mechanisms/attachment members, as further detailed below. Exemplary connecting/attachment members include such mechanisms as, but not limited to: magnets, vacuum, “pig-tail” structure, lasso structure, a loop structure, a net, a basket structure or any combination thereof. In some embodiments, the stylet may further include or be functionally associated with an indication unit configured to provide an indication of a connection between the attachment member of the stylet and the connecting section of the indwelling stent. An exemplary indication unit (164), located at handle (167) in the external section (165) of stylet (160) is illustrated in FIG. 4B. Further shown is string (139), connected between external end (132) of intermediary catheter (130) and external end (165) of stylet (160). String (139) may be in a relaxed position (as shown in FIG. 4B) or in a stretched position (FIG. 4C, below), depending on the location of stylet (160) within intermediary catheter (130). String (139) may be used for the maneuvering of the stylet and/or to control/limit the extent that the stylet can be pulled out of the intermediary catheter, once the stylet is connected to the indwelling catheter, to thereby serve as a securing/stopper member. Once connected to the indwelling stent (144), stylet (160) may be operated by a user (for example, by using handle (167)), to maneuver the indwelling stent and to place the connecting section thereof inside the tip section lumen region (134) of intermediary catheter (130), so as to form a secure, continuous fluid passage/conduit between intermediary catheter (130) and indwelling stent (144). Thus, when stylet (160) is proximally retracted (towards the external end) within the inner lumen of intermediary catheter (132), the connecting end of indwelling stent (144) is pulled into the inner lumen of the intermediary catheter. As shown in FIG. 4C, when stylet (160) is proximally retracted within the inner lumen of intermediary catheter (132), string (139) acquires a stretched position, which limits the further pulling of stylet (160) out of the intermediary catheter. By this mode, the string serves as a securing/stopper member which secures the connection between the indwelling stent and the stylet, within the inner lumen of the intermediary catheter.

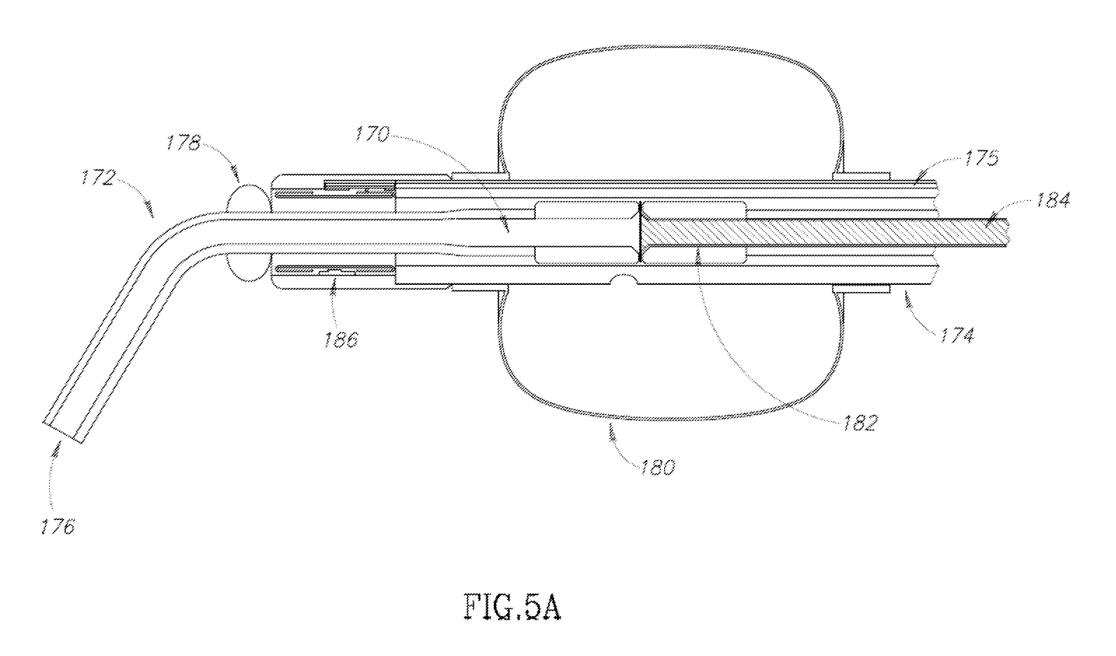

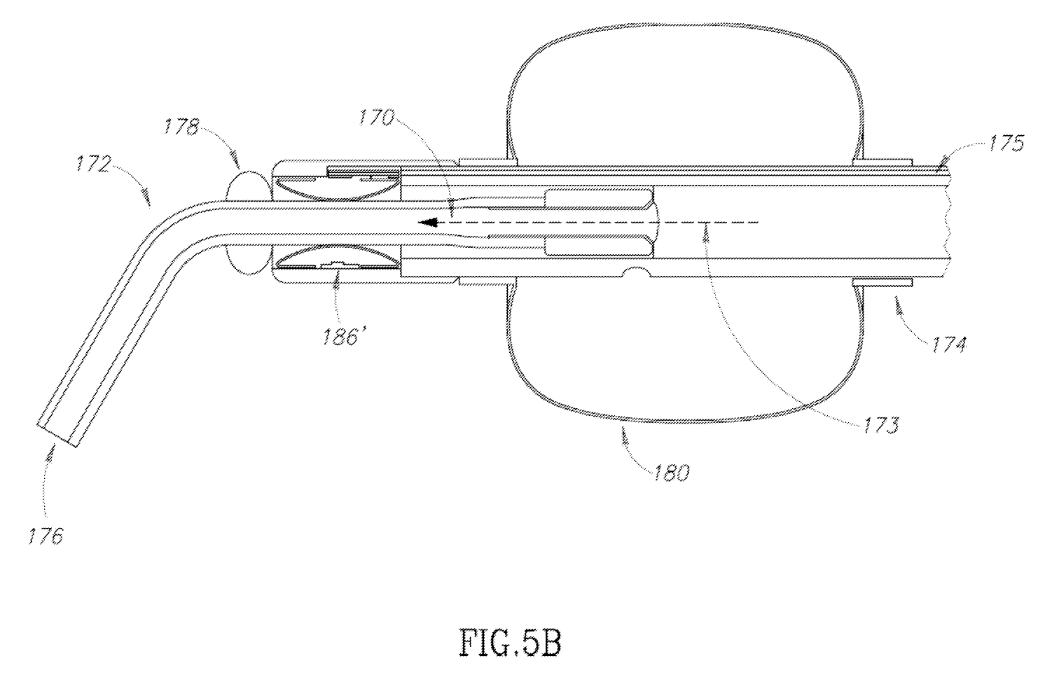

Reference is now made to FIGS. 5A-D which show close-up views of a cross section of the tip section of an intermediary catheter being connected to an indwelling stent, according to some embodiments. As shown in FIG. 5A, connecting section (170) of indwelling stent (172) is located within the inner tip section lumen of intermediary catheter (174), while the target section (176) of the indwelling stent (172) is not inserted into inner lumen of intermediary catheter. As further shown in FIG. 5A, indwelling stent (172) includes a stopper element (shown as stopper element 178). The stopper element may be located at the outer wall of the indwelling stent at a location between the connecting section and the target section. In some embodiments, the stopper element is configured to determine the portion (such as, length) of the indwelling stent that can enter the inner lumen of the intermediary catheter, thus locating the securing member and sealing member on the intermediary catheter in an accurate position relative to the indwelling stent for best functionality. In some embodiments, the indwelling stent may have one or more regions having special shape(s), such as an increased diameter that may increase the holding power of an inflated inner balloon and increase securing and sealing of the connection. In some embodiments, the stopper element may reside on the entire circumference of the indwelling stent or on portions thereof. In some embodiments, the stopper element is a stopper ring. Further shown in FIG. 5A is an anchoring element (shown as anchoring balloon 180) configured to anchor intermediary catheter (174) to its location within the subject's body. Further shown are the connecting section (170) of the indwelling stent (172) connected to connecting member (182) of stylet (184) that has been inserted via the internal lumen of the intermediary catheter (174), as detailed above. Additionally, on the inner walls of the tip section of the intermediary catheter, a securing member (186) is located. The securing member may be configured to secure the connecting section of the indwelling stent within the inner lumen of the tip section of the intermediary catheter. The securing member may be deployable and may include one or more inflatable balloons that may be inflated, for example, via an inflation lumen (175), which may be located adjacent to the internal lumen of the intermediate catheter (for example, in the inner walls of the intermediate catheter), using, for example, a syringe connected to a connector located on the external section of the intermediary catheter. In some embodiments, the securing member may include any one of securing means, such as, but not limited to: a securing balloon, an internal stopper ring, one or more recesses on the inner walls of the tip section of the intermediary catheter and/or on the outer walls of the connecting section of the indwelling stent, vacuum means, magnets, an internal or external locking mechanism such as a rod inserted through the intermediary catheter that can be rotated to engage a recess on the magnet or other part of the indwelling stent, and the like, or combinations thereof. In some embodiments, the securing member is further used to seal the connection between the indwelling catheter and the intermediate catheter so as to form a continuous fluid conduit. In some embodiments, the system may further include a sealing element/member that may be used to seal the connection between the indwelling catheter and the intermediate catheter, so as to form a continuous (leakage free) fluid passage (conduit). In some embodiments, the sealing member is in addition to the securing member. In some embodiments, the sealing member is placed instead of the securing member. In some embodiments, the system further includes means/controllers for identifying and verifying the connection between the intermediary catheter and the indwelling stent and/or verification of the formation of a continuous catheter having a continuous fluid passage there within. In some embodiments, the verifications means may include such means as, but not limited to: electric impedance sensor, internal air pressure sensor, magnetic proximity sensor, fluid pressure sensor, and the like, or any combination thereof.

Reference is now made to FIG. 5B, which shows the formation of a continuous catheter enabling a continuous passage of fluids and other materials, according to some embodiments. As shown in FIG. 5B, securing member (186′) has obtained an inflated position, while securing the connecting section of the indwelling stent (170) into its location within the inner lumen of the intermediary catheter. As further shown in FIG. 5B, the stylet has been removed, and a continuous fluid passage (conduit) is formed, allowing transfer of materials from/to the proximal (external) end of the intermediary catheter, via the tip section thereof, the connecting section of the indwelling stent and to/from the target section of the indwelling stent. The exemplary fluid flow is represented by dashed lines (173). The materials transferred through the continuous catheter may be applied/removed/injected via a connector on the external section of the intermediary catheter. After the desired materials have been transferred to the designated locations, the indwelling stent may be disconnected from the intermediary catheter. The disconnection of the indwelling stent from the intermediary catheter may be performed by various means. For example, a guide rod (such as mandrel or guide wire) may be inserted thought the external section of the intermediary catheter, via the internal lumen of the intermediary catheter, to push the indwelling stent away from the intermediary catheter. The disconnection of the indwelling stent may be performed after disengaging the securing member which secures the connecting section of the end of the indwelling stent within the tip section region of inner lumen of the intermediary catheter. In some embodiments, the disconnection may be achieved by use of external magnets, capable of separating the connecting section of the indwelling stent from the tip section of the intermediary catheter. In some embodiments, even a small separation between the intermediary catheter and the indwelling stent is sufficient to enable the removal of the intermediary catheter without displacing the indwelling stent.

As shown in FIG. 5C, which is a close up view of a cross section of the tip section region of intermediate catheter (174), the connecting section (170) of indwelling stent (172) is located within the inner tip section lumen of intermediary catheter (174). The connecting section includes an attachment member (193), shown in the form of a hollow magnet, which is connected to a corresponding attachment member (192) located at the tip section of stylet (190). Further shown is deployable securing member (191). Securing member (191) may serve as a deployable securing member that is configured to secure the connecting end (170) of indwelling stent (172) within the inner lumen of intermediary catheter (174) and/or to further provide a sealing of the connection between the indwelling stent and the intermediate catheter lumens. As shown in FIG. 5D, the stylet has been removed from inner lumen (196) of intermediate catheter (174), and securing member (191′) has acquired an inflated formation, thereby sealing the connection between indwelling catheter (172) and intermediate catheter (174) and forming a continuous fluid passage (conduit), represented by dashed arrow (194).

As detailed above, the approximation/connection/attachment between the connecting section of the indwelling stent and the tip section of the intermediary catheter may be achieved by various means. For example, magnets with opposing polarities may be mounted on the connecting section of the indwelling stent and on the tip section of the intermediary catheter, such that when the two ends are in close proximity, they may magnetically attract and connect. For example, structures configured to attach to each other may reside on the respective ends of the indwelling stent and the intermediary catheter. Such structures may include such structures as, but not limited to: lasso, “pig-tail”, net, basket structure, and the like. For example, vacuum ducts located on the tip section of the intermediary catheter may be used to attract the connecting section of the indwelling stent.

According to some embodiments, the system may further include a verification member, configured to verify that the components of the system can specifically interact and connect and form a secure, continuous catheter. In some embodiments, the verification member allows the formation and/or operation of the continuous catheter system. In some embodiments, the connecting section of the indwelling catheter and/or the tip section of the intermediate catheter and/or the tip section of the stylet (or the attachment member thereof) include a verification member. In some embodiments, the verification member is an RFID element, configured to identify a corresponding member on a corresponding interacting part. The use of such verification member provides an added safety means to ensure that the system parts are compatible and can indeed securely and safely connect within the subject body.

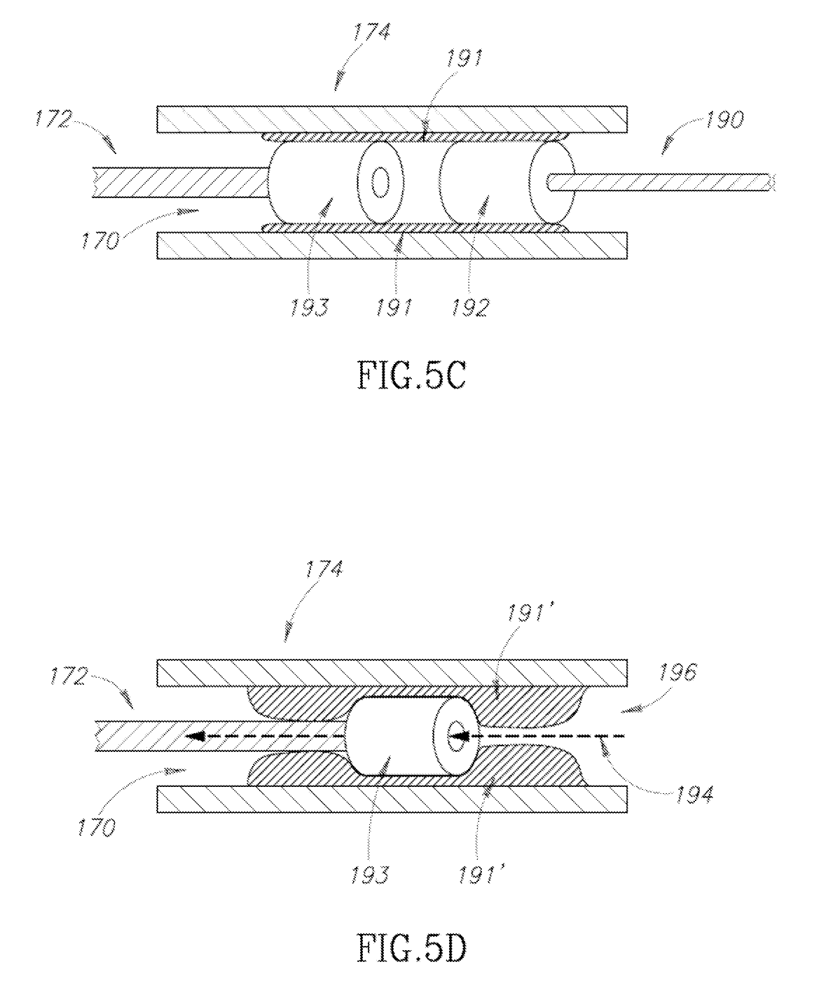

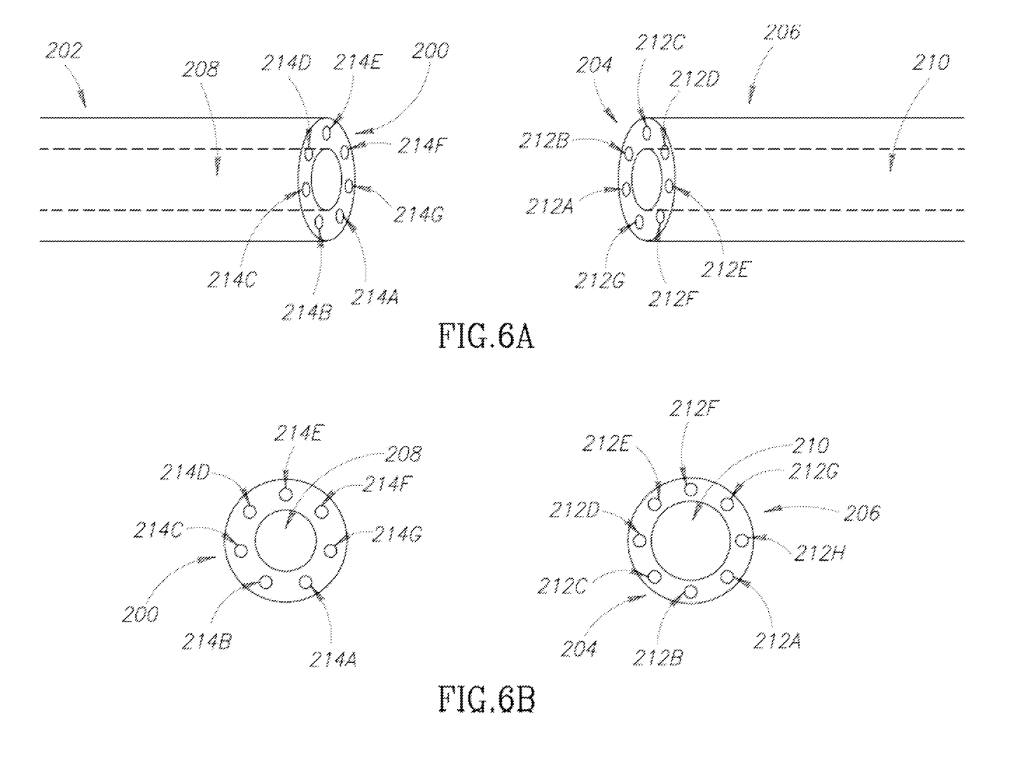

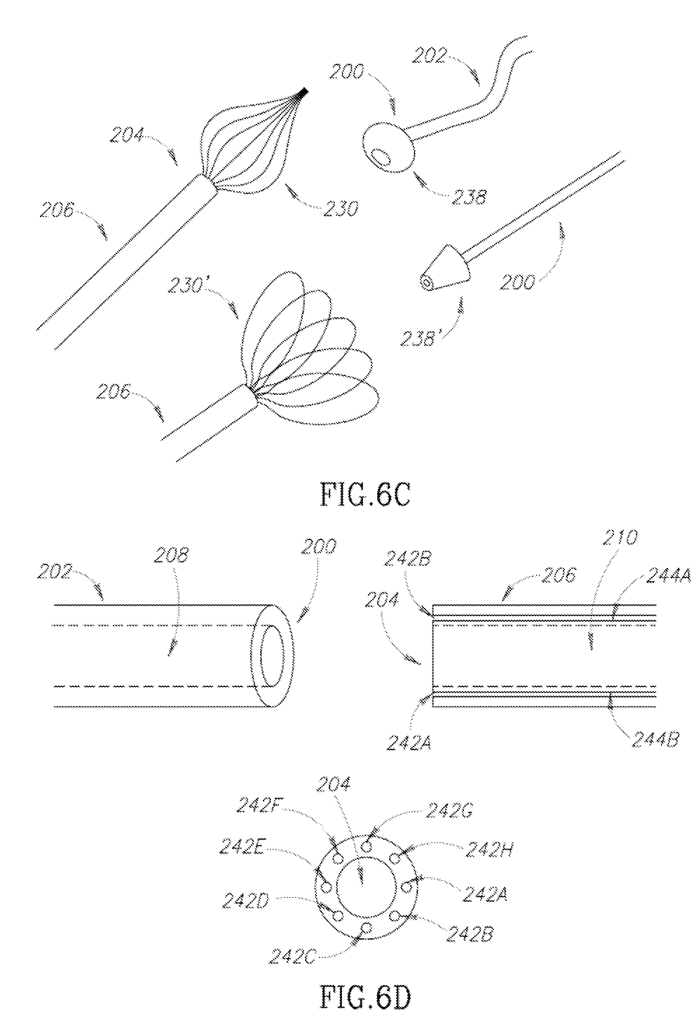

Reference is now made to FIGS. 6A-D, which show schematic illustrations of exemplary means for approximating and/or connecting and/or securing the connection of the tip section of an intermediary catheter with the connecting section of the indwelling stent, according to some embodiments. FIG. 6A, shows a cross section of the connecting section (200) of the indwelling stent (202) and the tip section (204) of the intermediary catheter (206), which include or are attached to magnets of opposite polarities (exemplary shown as hollow cylindrical magnetic tips, (212A-G) on tip section (204) of intermediary catheter (206) and magnetic tips (214A-G) on connecting section (200) of indwelling stent (202). Further shown are internal lumen (208) of indwelling catheter (202) and internal lumen (210) of intermediary catheter (206). FIG. 6B shows a front view of the respective sections presented in FIG. 6A. When the magnetic tips (212A-G) of tip section (204) of intermediary catheter (206), is in proximity to the corresponding magnetic tips (214A-G) of the connecting section (200) of indwelling stent (202), the magnetic attraction causes the tips to attach to one another, thus creating a continuous conduit (shaft/passage/channel) through the lumens (208, 210) of the respective indwelling stent (202) and the intermediary catheter (206) and the hollow magnets connecting them. Alternatively, magnetic particles may be present on one of the tip section of the intermediary catheter or the connecting section of the indwelling stent and magnetizable particles may be present on the other component. In some embodiments, means may be provided to ensure an end to end attachment of the two catheters and prevent a side by side attachment. For example, an inflatable balloon (not shown) may be present around the tip section of the intermediary catheter lumen (210) which prevents the connecting section of the indwelling stent from forming a side by side attachment to the lumen 210.

As shown in FIG. 6C, a three-dimensional basket structure that may have an open/closed position may be used for approximating and attaching the respective ends of the indwelling and the intermediary catheter. As shown in the top panel of FIG. 6C, the basket (230) is in a closed position, and is attached or is integral to the tip section (204) of the intermediary catheter (206). The connecting section (200) of the indwelling stent (202) has a corresponding structure (238), configured to attach or be inserted into the basket. As shown in the bottom panel of FIG. 6C, the basket (230′) is shown in an open position (for example, after being inserted into the body and placed in the corresponding cavity/lumen). The open basket (230′) may then close over the indwelling stent connecting section that is structured or attached to a structure (238′), which can provide an adequate anchor for the basket.

According to some embodiments, after the respective ends of the intermediary catheter and the indwelling stents are attached, the indwelling stent may be pulled into the internal distal (tip section) lumen (shaft) of the intermediary catheter. For example, if magnets were used to attach the respective ends, both magnets would enter that shaft. In the case that the magnets are attached side-by-side, entering the shaft will either force them to re-align to face-to-face attachment or they will get detached and the process of attraction and attachment can be retried. When the magnets are attached inside the shaft, a securing/locking mechanism may be applied to secure/lock their attachment. In some embodiments (as exemplified above herein), the magnets may be pulled into the shaft toward a specific position and stopped, for example, by use of a stopper element, such as stopper ring (as exemplified in FIG. 5A), or an external string (as exemplified in FIGS. 4B-C). When the magnets are in position, an internal anchoring/securing element (such as internal anchoring balloon) may be inflated to lock the magnets together. Inflation of such balloon may be achieved, for example, by connecting a source of liquid or gaseous fluid (such as a syringe filled with sterile saline), to a connector located on the external section of the intermediary catheter. Alternatively or additionally, a vacuum seal may be formed between the respective ends of the intermediary catheter and indwelling stents. As shown in FIG. 6D, one or more vacuum ducts (shown as exemplary ducts 244A-B) may extend along the wall of an internal lumen (210) of the intermediary catheter (206), that terminate at the external end of the lumen in a connector (not shown) which can be connected to a source of negative pressure. The vacuum ducts may terminate in orifices, (shown as exemplary orifices 242A-H) in tip section (204) of intermediary catheter (206). When the respective ends of the intermediary catheter and indwelling stents are in proximity to each other in the subject's body, the ends are drawn towards each other by the vacuum created in the vacuum ducts to form a stable fluid-tight connection.

According to some embodiments, there is thus provided a connectable catheter system, comprising: an intermediary catheter comprising an external section and a tip section, the tip section is configured to be inserted into a body of a subject; an indwelling stent, configured to be located within the body of the subject, the indwelling stent comprises a connecting section and a target section; wherein the connecting section of the indwelling stent is configured to reversibly connect, within the body of the subject, to the tip section of the intermediary catheter to form a continuous conduit from the external section of the connectable catheter to the target section of the indwelling stent.

According to some embodiments, the indwelling stent may be designed to have tip articulation and manipulation capabilities that enable increased control over the locale of fluids passage (fluid target location). In some embodiments the tip articulation is implemented by one or more (such as, two or more) strings that are inserted through the indwelling stent lumen such that pulling any of the strings may change the angle and direction of the target section of the indwelling stent. Combining the change of direction, the target section of the indwelling stent and the controlled pulling or pushing enables relocation of the indwelling stent target section essentially to any desired location in the target cavity. In some embodiments, the articulating strings are connected within the attachment member of the indwelling stent to the intermediary catheter. In some embodiments the articulating strings are passed externally to the indwelling stent, through the body cavity to outside the subject's body.

In some embodiments a ring of radio-opaque pigment is inserted over the catheter tip section to enable the tracking of the tip location by an external x-ray system.

In some embodiments, the intermediary catheter may be provided with a radio-opaque tip in order to locate the tip in radiographic images.

In some embodiments the tracking of the tip location is enabled by a miniature camera device connected to the catheter tip section. Such cameras, with typical pixel dimension of between 1 to 2.5 micro-meter are available in matrixes that are small enough, for example 100 micro-meters length, to be inserted via an indwelling stent.

According to some embodiments, the target section of the indwelling stent may be shaped or formed in one or more loops to anchor it and retain its position within the target cavity. In some embodiments a string may be attached to the anchoring loop in a way that when the string is tightened, it secures the anchoring loop to prevent opening thereof under pull forces, which may result in misplacing the correct positioning within the target body cavity. This string can be a lumen and may be connected to the target section. In some embodiments the string can be one of the strings that are used for articulation of the tip of the indwelling stent. In some embodiments, the string can have internal tension such that it would acquire an open position when released externally from tightening when inserted through an internal lumen in the indwelling stent.

In some embodiments, alternative anchoring mechanisms may be used for retaining the position of the indwelling stent in its target location, such as, for example, a Nitinol rod inserted into the target section of the indwelling stent, which may strengthen the loop holding force.

According to some embodiments, the connecting section of the indwelling stent is configured to fit into an inner lumen of the tip section of the intermediary catheter.

In some embodiments, the inner lumen of the intermediary catheter includes a securing member configured to secure the connecting section of the indwelling stent within the inner lumen of the tip section of the intermediary catheter. In some embodiments, the securing member is deployable. In some embodiments, the securing member may include one or more inflatable balloons. According to some embodiments, the intermediary catheter may further include an inflating port/connector configured to inflate the one or more inflatable balloons. In some embodiments, the securing member includes vacuum ducts. In some embodiments, the securing member is mechanical and includes corresponding recesses or grooves in the respective catheter ends. In some embodiments, the mechanical securing member includes matching interacting hooks, capable of securing the catheters ends. In some embodiments, the securing member includes a flexible plate (leaf). In some embodiments, the flexible plate is made of metal. In some embodiments, when an inner securing balloon is used, the flexible metal plate may be fitted on top of the inner securing balloon, such that it may move into the path of the connecting section of the indwelling catheter, when the securing balloon is inflated, thus creating a mechanical lock that secures the connection section of the indwelling stent, within the intermediary catheter tip section (within the inner lumen thereof). In some embodiments, the securing member is configured to reversibly secure the connecting section of the indwelling stent within the inner lumen of the tip section of the intermediary catheter. According to some embodiments, more than one securing member (identical, similar or different) may be configured to act in parallel. According to some embodiments, the securing member may be further configured to seal a fluid passage between the outer wall of the indwelling stent and the inner wall of the indwelling stent, so as to form a continuous conduit, with no leakage of fluid passing therein. In some embodiments the sealing member is a separate member, capable of sealing a fluid passage between the outer wall of the indwelling stent and the inner wall of the indwelling stent, which is placed in addition to or alternatively to the securing member. In some embodiments, the sealing member is deployable.

According to some embodiments, the catheter system may further include a stylet removably insertable into the inner lumen of the connectable catheter. In some embodiments, the stylet may include, at a tip section thereof, an attachment member configured to attach to the connecting section of the indwelling stent end, such that when the stylet is proximally retracted within the inner lumen of the connectable catheter the indwelling stent is pulled into the inner lumen of the connectable catheter. In some embodiments, the stylet's attachment member may include a magnet configured to attract/approximate the connecting section of the indwelling stent. In some embodiments the stylet tip section may be bent to enable it to be attracted to the corresponding attachment members (such as magnets) of the connecting end of the indwelling stent, that are located below the stylet insertion path. In some embodiments, the attachment member (such as a magnet) is connected to the stylet tip flexibly to enable it to be attracted to magnets that are located behind the stylet insertion point out of the intermediary catheter tip and into the body cavity. In some embodiments, the stylet may be used for searching/locating the indwelling stent within the subject's body, without a need to visualize the indwelling stent. In some embodiments, the stylet may be used to attract, attach, connect and/or pull the indwelling stent into the inner lumen of the intermediary catheter. In some embodiments, the stylet tip section is integrally formed with the attachment member. In some embodiments, the attachment member of the stylet is attached or connected (temporary or permanently) to the tip section of the stylet. In some embodiments, the attachment member of the stylet is functionally associated with the tip section of the stylet. In some embodiments, the stylet's tip section (and/or the attachment member) is flexible and may bend in any direction, so as to allow the finding, attaching and/or connecting of the connection section of the indwelling stent within the subject's body. In some embodiments, the stylet tip section (and/or attachment member, if not integrally formed therewith) may be made of nitinol or other memory-material, which may acquire a bent or flexible shape when protruding through the tip section of the intermediary catheter. In some embodiments, the stylet may further be configured to provide an indication once a connection has been made to the connecting section of the indwelling stent. In some embodiments, the stylet's attachment member may include a loop, lasso, pigtail, net, basket structure or any combination thereof. In some embodiments, the connecting section of the indwelling stent is fitted with a corresponding connection member that may be as an integrated part of the stent luminal tube. In some embodiments, the stylet may further include one or more internal conduits, capable of transferring fluids, wires, connections, and the like from an external region to the internal region.

According to some embodiments, the connecting section of the indwelling stent may include a magnet or a component attractable by the stylet's magnet.

According to some embodiments, the stylet's attachment member may include a loop, lasso, pigtail, net, basket structure or any combination thereof, capable of attracting, attaching and/or connecting to the connecting section of the indwelling stent.

According to some embodiments, the stylet may further include or be functionally associated with an indication unit configured to provide an indication of a connection between the attachment member of the stylet and the connecting section of the indwelling stent. The indication may include any type of indication, such as, tactile indication, visual indication and/or audible indication.

According to some embodiments, the indwelling stent may further include a stopper element at an outer wall thereof, wherein the stopper element is located between the connecting section and the target section and wherein the stopper element is configured to determine the portion (length) of the indwelling stent that can enter the inner lumen of the connectable catheter. In some embodiments, the stopper includes a measured length of string connected between the stylet handle and a connector, fixed on the stylet that fits an instillation port of the intermediary catheter. After the stylet is inserted into the intermediary catheter, the connector is closed/locked on the instillation port. After the stylet attachment member (for example, magnet) is attached/connected to the indwelling stent connecting section, the string is limiting the extent that the stylet can be pulled out of the temporary catheter, so that the coupled magnets are brought to the exact position, to ensuring sealing and securing thereof within the lumen of the intermediary catheter.

According to some embodiments, the connectable catheter system may further include a guide rod (such as a mandrel or guide wire) which may be removably insertable into the inner lumen of the connectable catheter. In some embodiments, the mandrel/guide wire may be configured to facilitate the penetration of the tip section of the connectable catheter into the body lumen.

According to some embodiments, the guide rod may be further configured to remove the indwelling stent from the connectable catheter when pushed distally within the inner lumen of the connectable catheter.

According to some embodiments, the intermediary catheter may further include a deployable anchoring element located at an outer wall thereof, the deployable anchoring element, when deployed, is configured to anchor the connectable catheter in the body lumen. In some embodiments, the deployable anchoring element is a balloon.

According to some embodiments, the intermediary catheter may further include one or more substance administration port(s)/connector(s) allowing the administration of the substance from the external section of the intermediary catheter to the target section of the indwelling stent.

According to some embodiments, the intermediary catheter may include more than one internal lumen. In some embodiments, the lumen(s) is configured to transfer fluids, substances or devices therethrough. In some exemplary embodiments, the intermediary catheter may include an additional internal lumen at a wall thereof. In some embodiments, an internal lumen may be as an inflation lumen, configured to inflate various internal members, such as, for example, but not limited to: securing member, sealing member, and the like. In some embodiments, the inner lumens may be parallel to each other and may be identical, similar or different in length, composition and/or diameter.

According to some embodiments, the system may further include a circuitry unit configured to provide various indications and/or controlling the operation of the system. In some embodiments, the control circuitry unit may be configured to provide indication when the respective ends of the intermediary catheter and the indwelling stent are attached/connected. In some embodiments, the control circuitry unit may be configured to provide indication when the continuous fluid passage is formed. In some embodiments, the circuitry unit is a control circuitry unit.

According to some embodiments, the system may further include a vacuum source configured to create a vacuum in the inner lumen of the connectable catheter to secure the positioning of the indwelling stent.

According to some embodiments, the system may further include means/members for heating and/or cooling a composition being instilled to the target cavity through the connectable catheter system, in order to control and vary the viscosity and other properties of the composition during instillation. In some embodiments, such member may be an external member. In some embodiments, such member may be an internal member. In some embodiments, such member may be internal and/or external.

According to some embodiments, following completion of the instillation/administration of a desired material, the respective ends of the indwelling stent and the intermediary catheter can be disconnected. In an exemplary embodiment, the connecting section of the indwelling stent may have a smaller internal diameter than the tip section of intermediary catheter. Separating these two parts, after the connecting magnets are pushed out of the intermediary catheter, can be achieved by inserting a guide rod or stylet into the internal lumen, while exerting a force on the stopper of the indwelling stent. In a non-limiting example, this guide rod or stylet can have a known length, or visible scale of length, or an external string connected thereto, that ensure magnets separation, while reducing the risk of damage to the internal cavities, such as perforation of the internal cavity. In some embodiments, the separation of the connecting magnets can be achieved by an external magnet that is placed externally in close proximity to the internal cavity while detracting the magnet at the end of the indwelling stent, while the intermediary catheter is pulled away. In some embodiments, the magnets may have longitudinal polarization and thus rotating the intermediary catheter may cause the magnets to realign in a way that detracts them from one another.

After separation between the intermediate catheter and the indwelling stent, the intermediary catheter may be removed from the body. The indwelling stent is thus retained in its internal location after removal of the intermediary catheter, and can be used again in subsequent uses. With the indwelling stent maintained in position, subsequent uses (such as instillations) may be performed without the use of x-ray guidance and possibly with just mild sedation, thus reducing radiation hazards, insertion trauma and anesthesia complications. For example, retaining the indwelling stent inside the urinary tract for subsequent instillations tends to prevent or minimize infection, irritation, sensitization and injuries to the ureter orifice that may be incurred by repeated instillations of ureteral catheter in the upper urinary tract. The outer surface of the indwelling stent may be coated with a hydrogel to reduce friction between the indwelling stent and body tissues during insertion and during the indwelling period. Risk of infection may be reduced by application of an antibacterial coating to the outer surface of the indwelling stent.

According to some embodiments, the system may include the sequential or parallel use of more than one intermediary catheter and/or more than one indwelling stent. In some embodiments, an indwelling stent may be connected sequentially to one or more intermediary catheters, such that in one instance a substance is being delivered/administered from an external location to an internal body location and in a second instance, a substance is being removed from the internal location to an external location. For example, circulation of a substance may be performed whereby the substance is administered via a first continuous catheter system and is being removed via a second continuous catheter system. This may be utilized, for example, for inserting a heated substance to a target location and removing the heated substance from the internal target location, while performing circulation of said substance within the internal target location. In some exemplary embodiments, the substance comprises a gel. In some embodiments, the substance is in a form of a gel.

In some embodiments, more than one connectable continuous catheter systems may be used. In such a system, for example two or more indwelling stents (that may be identical, similar or different) may be connected to corresponding two or more internal lumens within the intermediary catheter. In such settings one or more stylets may be used. For example, one stylet may be used to attach and/or connect and/or attract a first indwelling stent and then the second indwelling stent. In some examples, two separate stylets may be used, each attaches to a separate indwelling catheter.

According to some embodiments, there is provided an intermediary (main) catheter, comprising: an external (proximal) section (end) and a tip (distal/internal) section (end), the tip section is configured to be inserted into a body lumen of a subject; a securing member located at an inner lumen of the intermediary (main) catheter, the securing member is configured to secure a connecting section of an indwelling stent within the inner lumen of the tip section region thereof and thereby to form a continuous fluid passage (conduit) between the inner lumen of the connectable catheter and an inner lumen of the indwelling stent. In some embodiments, the securing member may be further configured to seal a fluid passage between an outer wall of the reconnectable indwelling stent and the inner wall of the intermediary catheter. In some embodiments, the intermediary catheter may further include a sealing member configured to seal a fluid passage between the outer wall of the reconnectable indwelling stent and the inner wall of the intermediary catheter.

According to some embodiments, there is provided an indwelling stent, comprising: a connecting section and a target section, the target section is configured to be positioned into a target body cavity of a subject; the connecting section is configured to reversibly connect, within a body lumen, to the tip section of an intermediary catheter; and a stopper element at an outer wall thereof, wherein the stopper element is located between the connecting section and the target section and wherein the stopper element is configured to determine the portion (such as, length) of the indwelling stent that can enter an inner lumen of the intermediary catheter.

In some embodiments, the indwelling stent, and/or the intermediary catheter may be made of medical grade biocompatible polymers that are resistant to aging, histocompatible, non-toxic and have smooth facials. One or both of the indwelling stent, and the intermediary catheter may be disposable or suitable for multiple uses.

According to some embodiments, the indwelling stent may further include X-ray markers at its tip section (radio-opaque lines) for fluoroscopy visualization.

In some embodiments, the indwelling stent may include one or more anchors, located at the target section, which allow the positioning and securing of the target section of indwelling stent in the target cavity. For example, one or more anchors formed from a shape memory alloy may be used to anchor the indwelling stent.

According to some embodiments, the system of the invention may be provided with means for generating an indication that the respective ends of the indwelling stent and the intermediary catheter are connected in a fluid-tight connection. For example, electrical contacts may be present at the tip section of the intermediary catheter internal lumen, which may form a closed circuit when a connection is formed. Closure of the contacts creates an electric current in a circuit which can be used as an indication that the fluid-tight connection has been formed. In another embodiment, a thin tube which conducts air as part of the internal lumen of the intermediary catheter may be used. In such embodiment, the tube path is designed so that the air pressure inside the tube is affected by the connection between the indwelling stent and the intermediary catheter. Changes in the tube internal air pressure indicate the formation of a connection and a level can be defined for indication of a fluid-tight connection. Additionally or alternatively, the pressure inside the locking balloon (as shown in FIG. 1) can be monitored and a predetermined pressure level can serve as indication that the attachment means (for example, magnets), are locked in position inside the lumen of the intermediary catheter.

In some embodiments, the locking of the connecting members (for example, magnets) can be achieved by a locking mechanism that is either designed as part of the internal lumens or is inserted through the intermediary catheter lumen and pulled against a distal magnet, thus creating a lock mechanism that requires no external indication to verify its integrity.

According to some embodiments, approximation, connection and/or attachment of the intermediary catheter and the indwelling stent (via, for example, a stylet), may be affected by a magnet. According to some embodiments, the steps of seeking and connecting between the indwelling device and the intermediate catheter may be achieved using a non-directional magnet interaction (being strong as it is non-directional), between these members (directly, or via, a stylet, for example). This may be followed by establishment of a functional contact within the catheter system, which may be implemented, in some embodiments, by a separate (tubular) element, equipped/attached/connected/glued/formed with a diametrically magnetized magnet. In some embodiments, such division of function is advantageous as it may allow a strong magnetic attraction between the stent and the intermediary catheter, separately from the actual directional attachment, which is affected by the diametrical magnets. Accordingly, in some embodiments, a directional connectivity between the system components (for example, indwelling device, and intermediate catheter) may be obtained. In some embodiments, to obtain a directional connectivity between the system components (for example, indwelling device, and the stylet of the intermediary catheter) diametrical magnets may be used. As is known, magnets are constructed with two opposing poles, North and South poles, corresponding to the earth's magnetic field. Opposing magnetic poles attract each other while identical magnet poles repel each other. A diametrically magnetized magnet is magnetized across its diameter such that the north pole is on one curved side and the south pole is on the opposite curved side.

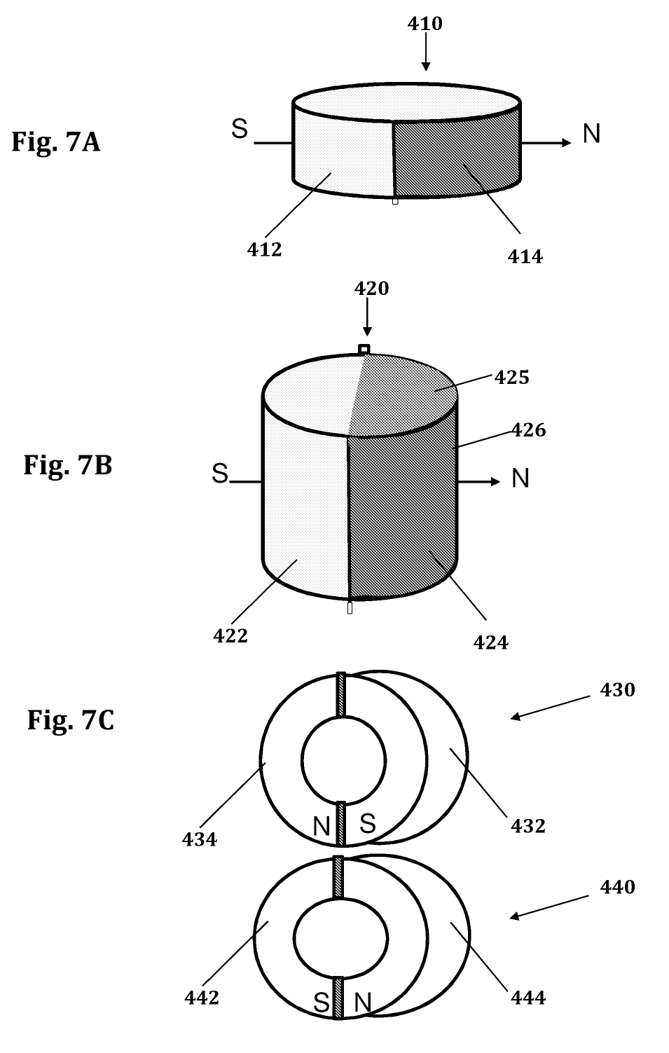

Reference is now made to FIGS. 7A-C, which show schematic illustrations of diametrical magnets, according to some embodiments. Shown in FIG. 7A is a perspective view of portion of a cylindered diametrical magnet (410). Diametrical magnet 410 has two opposing magnetized regions—South (S) pole region (412) and North (N) pole region (414). Cylinder magnets, such as magnet 420 in FIG. 7B can be magnetized axially or diametrically, such that a south pole region (422) and a north pole region (424) are obtained. When magnetized axially, the strongest points/regions of magnetic fields can be found on their flat, end-surface (425). When magnetized through their diameter, the strongest points/regions of magnetic fields can be found on their curved surface (426). FIGS. 7A and 7B depict diametrically magnetized magnets, i.e. magnetized through their diameter, with strongest points/strongest magnetic field on their curved surface. When placing two such diametrically magnetized cylinders face to face, they will orientate themselves so that opposing poles are attached together, as illustrated in FIG. 7C, which shows magnets 430 and 440 orient relative to the other such that the north side (434) of magnet (430) faces the south side (442) of magnet (440) and the south side (432) of magnet (430) faces the north side (444) of magnet (440). This will ensure that the positioning/orientation of the magnets relative to each other is maintained.

According to some embodiments, diametrical magnets may be embodied is one or more components of the catheter system disclosed herein, to achieve appropriate orientation of the various components, to ensure an accurate connection and further allow formation of one or more functional conduits/channels/lumens. In some embodiments, diametrical magnets may be used to connect a stent member (an indwelling device) and the stylet member of the intermediary catheter in a predetermined orientation, thereby enabling contact and/or connectivity of features designed around the cylinder circumference of such members. Such features may include, for example, lumens, holes, shafts, passages, channels, conduits, and the like, that can be used for various purpose, such as, for example, balloon inflation, conduit for fluid, such as, liquid flow, conduit for electrical contacts (for transferring sensors, motor electrical leads, heating elements), for mechanical connection (for example, for tip articulation and maneuvering), and the like, or combinations thereof. Each possibility is a separate embodiment.

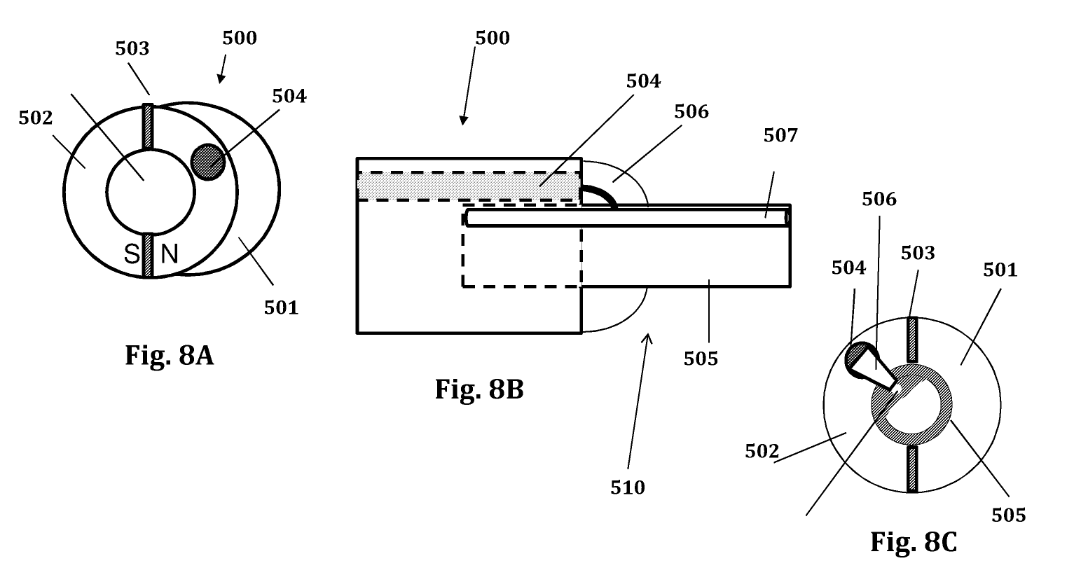

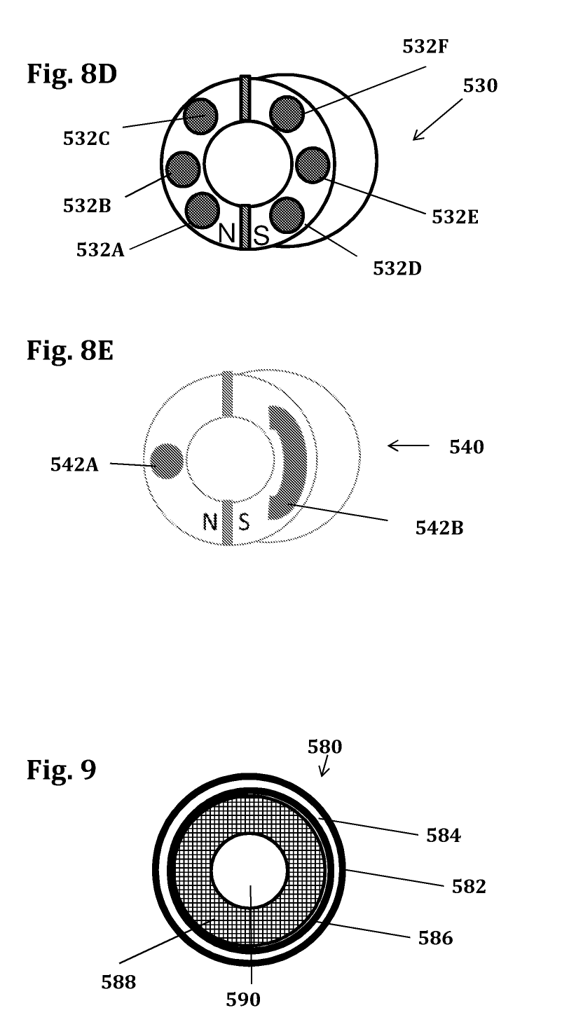

Reference is now made to FIGS. 8A-8C, which schematically illustrate top (FIG. 8A), side (FIG. 8B) and bottom (FIG. 8C) views of exemplary diametrical magnets allowing directional connection, according to some embodiments. Such directional connection enables control over the orientation of connection between various catheter system elements. Shown in FIGS. 8A-C, a magnet connection member (500) of tip of an intermediary catheter (510). Elements 501 and 502, represent the magnetic south and north poles, respectively, of a cylindrical diametrical magnet (500), separated by an imaginary boundary (503). The poles would orient so that they attract each other, when facing a corresponding diametrical magnet. Element 504 represents a lumen/channel/passage (“magnet conduit”), within the diametrical magnets, spanning from end to end (face to face) of the magnet. In some embodiments, the magnet conduit may be in any desired shape, form, size and/or diameter and may any desired physical, mechanical and/or chemical properties. In some embodiments, as further detailed below, the magnet conduits may include more than one conduit, wherein the conduit may be identical, similar or different is size, shape, form, composition or for the purpose for which it is intended. Each possibility is a separate embodiment.

In some embodiments, the magnet may be made or produced by sintering of metal powders and can be shaped to include one or more magnet conduits of any shape, form, size and/or dimension. In some embodiments, the magnets may be magnetized after the sintering process.

As shown in the side schematic view of FIG. 8B, tube/shaft 505 is inserted/located in the hollow center (508) of the diametrical magnets. Further shown is internal lumen (“inner conduit”) (507) within tube (505), and a connection (shown as electrical connection, 506), between magnet conduit 504 and inner lumen 507. FIG. 8C is a bottom schematic view of the directional connection of the diametrical magnets shown in FIGS. 8A and 8B. In some embodiments, a tube, such as, tube 505 may a catheter tube and may be made of various materials, such as, plastic. The plastic tube can be extruded to contain any number of longitudinal inner lumens/conduits that may have any required shape and internal diameter, starting at, for example, 0.1mm In some embodiments, the diametrical magnets, may be reversibly or irreversibly attached/connected/affixed/mounted/formed with the plastic elements. In some embodiments, the magnets may be attached/connected/affixed to the plastic tube by various means, such as, for example, mechanical means, including gluing, fusing, or molding. According to some embodiments, flexible or rigid elements, such as, metal rods, may be inserted into the inner conduits of the tube. In some embodiments, the inner conduits of the tube may be functionally or physically be connected or be associated with the magnet conduits. In some embodiments, the inner conduit of the tube may be connected to the magnet conduit using electrical wire. In some embodiments, the inner conduit of the tube maybe connected to the magnet conduit using pre-shaped tubes made of plastic or metal. In some embodiments, the connection between the tube internal conduits and the magnetic conduits allows the formation of multiple transfer conduits that may be used to function simultaneously and synergistically. For example, the connection between the tube conduits and the magnetic conduits may be used to enable liquid-transfer conduits along with electricity transfer liquid, for use in various settings, such as, for example, for hyperthermia by circulation of heated liquid. In some embodiments, the ability to connect magnetic conduits and inner conduits can allow mechanical connections over the magnetic connection.

According to some embodiments, as mentioned above herein, the diametrical magnets may include more than one magnetic conduit, wherein the magnetic conduit may be identical, similar or different in shape, form, size, dimension, composition, location, and the like. FIGS. 8D and 8E, to which reference is now made show schematic views of exemplary magnets having more than one magnetic conduits. Exemplary magnet 530 in FIG. 8D, includes 6 magnetic conduits (magnetic conduits 532A-F), which may be similar in shape, but may differ in size (diameter), composition, physical properties, mechanical properties, and the like. The magnetic conduits may be evenly spaced along the circumference of the magnet, having three magnetic conduits (532A-C) on the north pole side and three magnetic conduits (532D-F) on the south pole side. In some embodiments magnetic conduits 532A-F may each serve for a different purpose, i.e, be dedicated for transfer of different substance/energy/information. In some embodiments, one or more of the magnetic conduits may serve the same purpose. Exemplary magnet 540 in FIG. 8E includes 2 magnet conduits, 542A-B, each located at different side, and each having a different shape and size. In some embodiments magnetic conduits 534A-B may each serve for a different or similar purpose.

Reference is now made to FIG. 9, which illustrates an exemplary radial connection, allowing an electrical connection in the connectable catheter system, according to some embodiments. Illustrated in FIG. 9, a schematic top view of a radial connecting member (580), which includes an outer radial insulation layer (582), surrounding a conductive layer (584). Further shown is inner radial insulation layer (586), surrounding a magnet (588). Further shown is lumen (590). In some embodiments, the widths (size) of the conductive and insulating layers may be determined based on the current being conducted and the mechanical tolerance. In some embodiments, more than one conductive layers may be implemented. In some embodiments, the relatively low conductivity of the magnet may be sufficient for electrical grounding. In some embodiments, the outer insulation layer may be used to provide electrical insulation and to provide bio-compatibility to the connecting member.

According to some embodiments, the capability of directional connections between the connectable catheter parts can advantageously provide and support various types of connections. The directional capabilities can support, for example, various internal conduits shapes and sizes aimed for different purposes (such as, transfer of fluid), various electrical connections (for example, DC or AC) as well as mechanical connections (for example, by insertion and removal of rods with hooks). In some embodiments, the various types of connections can advantageously be initiated and supported separately, independently, or in parallel

According to some embodiments, using a directional connection in the connectable catheter system can advantageously allow various functions that can be implemented, based on the connection type. For example, utilizing a mechanical connection type can be implementable for tip articulation, i.e., guidance and control of the spatial location and positioning of the catheter tip in target areas. For example, utilizing liquid conduit connection type can be implemented for heating of internal regions/cavities by, for example, liquid circulation, as further detailed below. For example, utilizing electrical connection type can be used for heating target regions/cavities, for example, by high frequency heating, resistive heating and/or by infrared LEDs (such as, for example in the frequency range of 700 nm to 1000 nm, that is known to have photo-dynamic therapy (PDT) effects). For example, utilizing a balloon inflation conduit, a connection type can be implemented for instillation of heated substances to target area/cavity.

According to some embodiments, there is provided a connectable catheter system comprising an intermediary catheter and an indwelling stent catheter, wherein upon directional connection, of the intermediary catheter and the indwelling stent catheter, within the subject body, one or more connection conduits are formed to allow corresponding connection types. In some embodiments, the connection conduit may be selected from: fluid conduit (such as, liquid conduit, gas conduit), electrical conduit (capable of transferring currents (AC or DC), mechanical conduit (capable of allowing physical passage of rods, and the like). The intermediary catheter includes an external (proximal) section (end) and a tip (internal /distal) section (end) opposing thereto, the tip section is configured to be inserted into a body of a subject. The indwelling stent, configured to be located within the body of the subject, includes a connecting section (end) and a target section (end); wherein the connecting section of the indwelling stent is configured to reversibly connect, within the body of the subject, with the tip section of the intermediary catheter to form one or more continuous conduits from the external section (end) of the intermediary catheter to the indwelling stent.

According to some embodiments, there is provided a connectable catheter system, comprising: an intermediary catheter comprising an external section and a tip section, the tip section is configured to be inserted into a body of a subject; and a reconnectable indwelling stent comprising a connecting section and a target section, the target section being configured to be located within a body of the subject, wherein the connecting section of the reconnectable indwelling stent is configured to reversibly and directionally connect, within the body, to the tip section of the intermediary catheter to form one or more continuous fluid conduits between the intermediary catheter and the reconnectable indwelling stent. In some embodiments, the directional connection is affected by diametrical magnets.

According to some embodiments, there is provided a method of connecting a connectable catheter system, the method comprising: attaching and/or drawing a connecting section of an indwelling stent into an inner lumen of the intermediary catheter; forming a directional connection between inner conduits within the indwelling stent and the intermediary catheter; securing the connecting section of the indwelling stent within the inner lumen of the tip section of connectable catheter, thereby forming one or more continuous conduits between the inner lumens of the connectable catheter and the inner lumens of the indwelling stent; and optionally utilizing a control circuitry unit, providing an indication when continuous conduits are formed.

According to some embodiments, the process of connecting the connecting section of the indwelling stent and the tip section of intermediary catheter (while both ends reside inside the subject's body) may comprise the steps of (i) seeking the connecting section of the indwelling stent, (ii) drawing the connecting section of the indwelling stent towards the tip section of the intermediary catheter, (iii) connecting the respective ends of the indwelling stent and the intermediary catheter, (iv) forming a directional connection between the inner conduits of the indwelling stent and the intermediary catheter; securing the respective ends of the indwelling stent and the intermediary catheter together, and (v) confirming the secure attachment by an indication of the secure attachment. The connection process may be performed with or without the use of vision guidance.

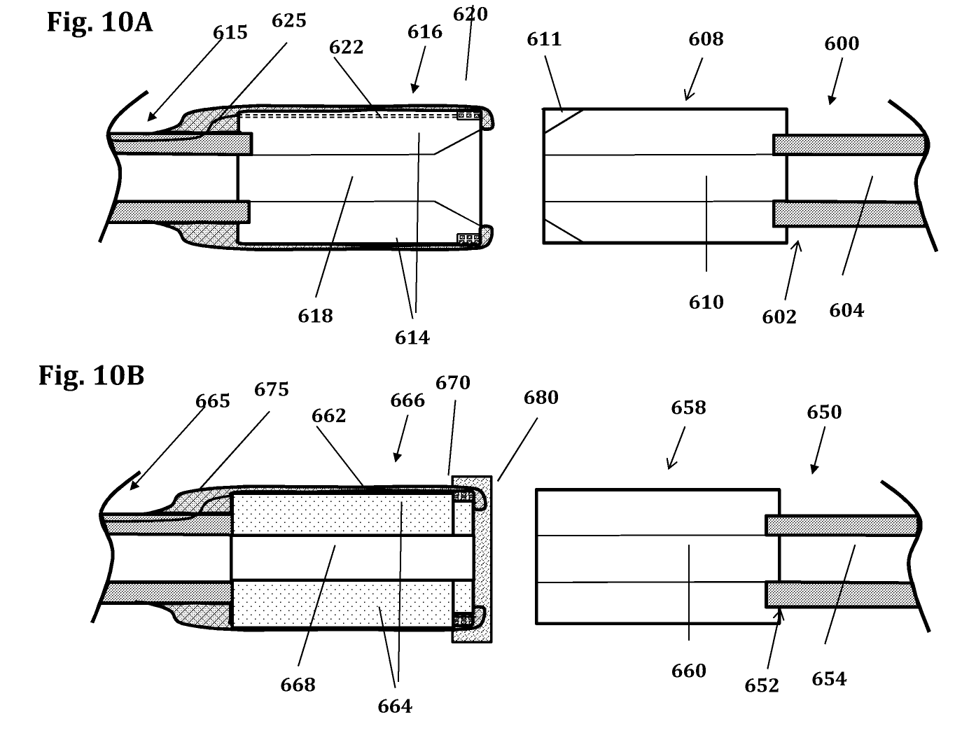

According to some embodiments, a direct stylet/intermediate catheter to indwelling catheter connection may be established, to allow formation of a continuous fluid conduit. Reference is now made to FIGS. 10A-B, which show schematic close-up views of a cross section of the tip sections of an intermediary catheter prior to being directly connected to the tip section of indwelling stent, using a magnetic connection, according to some embodiments. As shown in FIG. 10A, an internal lumen (602) of tip section (604) of indwelling stent 600, is attached to magnet (608) having a magnet conduit (610), which is continuous with the internal lumen of the stent. The magnetic attachment force is designed to overcome the pressure induced by the flow of liquid through the main lumen at the magnets connection region. As shown in FIG. 10A, magnet 608 has a slanted head (611). The end face (612) of magnet (608) can reversibly mate/attract to a corresponding magnet (614), which is attached to a stylet (615) within the connecting tip of intermediary catheter 616. Magnet (614) includes a magnet conduit (618), which is configured and aligned at one end with a stylet of the intermediary catheter and at its other end configured and aligned to reversibly mate with the slanted head 611 of magnet 608 of the stent. Further in the depicted embodiment, at the distal end of the intermediary catheter 616, a sensor coil 620 is in electric communication via sensor conduit 622 to an external controller unit (not shown). The sensor coil's wires (625) pass through sensor conduit (622), which is located within magnet (614) or pass adjacently to the circumference of magnet 614. In such a setting, when the stent is correctly positioned in the subject's body, an electrical (electronic) signal from the controller (not shown) can disconnect the magnet and the stent would separate from the intermediary catheter of the system, leaving the intermediary catheter securely connected to the indwelling stent, while forming a continuous fluid passage.

Shown in FIG. 10B, is a similar embodiment, in which the magnet of the indwelling catheter tip does not have a slanted head and the connecting tip of the intermediary catheter has a sealing gasket at the magnet face. As shown in FIG. 10B, an internal lumen (652) of tip section (654) of indwelling stent 650, is attached/connected/associated with magnet (658) having a magnet conduit (660), which is continuous with the internal lumen of the stent. The end face (662) of magnet (658) can reversibly attract with a corresponding magnet (664), which is connected with a stylet (615) within the connecting tip of intermediary catheter (666), which has a corresponding sealing gasket (680) on its connecting face, to ensure a sealed connection with the indwelling catheter. Magnet (664) includes a magnet conduit (668), which is configured and aligned at one end with a stylet of the intermediary catheter and at its other end configured and aligned to reversibly mate with magnet 658 of the stent. Further, at the distal end of the intermediary catheter 666, a sensor coil 670 is in electric communication via sensor conduit 672 to an external controller unit (not shown). The sensor coil's wires (675) pass through sensor conduit (672), which is located within magnet 664 (or passes adjacently to the circumference of magnet 664). In such setting, when the stent is correctly positioned in the subject body, an electrical signal from the controller (not shown) can disconnect the magnet and the stent would separate from the intermediary catheter of the system, leaving the intermediary catheter securely connected to the indwelling stent, while forming a continuous fluid passage.

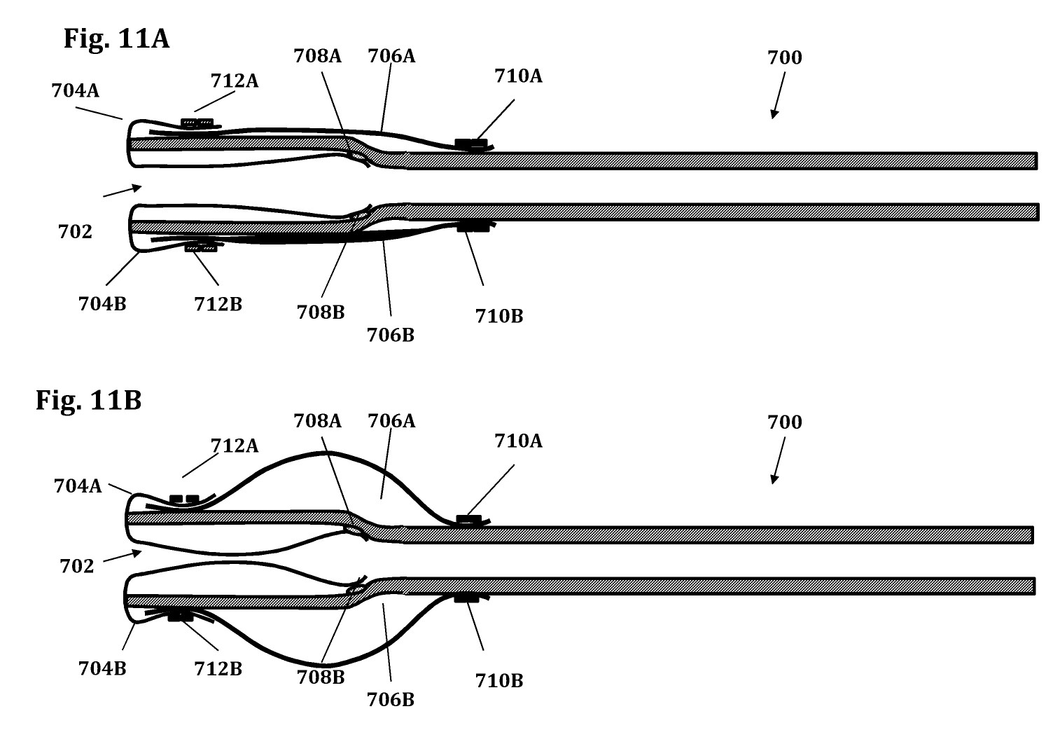

According to some embodiments, in order to improve catheter insertion and improve patient tolerability and compliance, the catheter tips (in particular, the intermediary catheter) may include/be covered with inner and/or outer balloons, to ease insertion into the subject's body. Reference is now made to FIGS. 11A and 11B which include schematic illustrations of cross sections of tip section of intermediary catheter, according to some embodiments. As shown in FIGS. 11A and 11B, intermediary catheter 700, includes at its connecting tip 702 (i.,e, the tip inserted into the subject body), inner balloons (704A-B) and outer balloons (706A-B), which essentially cover the ends of the catheter tube. The inner and outer balloons are shown in their deflated state in FIG. 11A and in their inflated state in FIG. 11B. The inner balloons are attached/bonded/glued to an internal region (708A-B) of the catheter lumen, and folded backward over the tip of the catheter. The outer balloons are pressed/attached/bonded/glued to an external region (710A-B) on the circumference of the catheter tube. At regions 712A-B, both internal and external balloons may be pressed/attached/bonded/glued to the circumference of the catheter tube.

In some embodiments, the inner and outer balloons may be similar or different in shape, size and composition. In some embodiments, the balloons may be made of polyurethane, silicon, rubber, and the like. In some embodiments, the inner balloons are made of polyurethane. In some embodiments, the outer balloons are made of silicon. In some embodiments, the folded proximal end of the inner balloons, the end near the catheter tip, and both ends of the outer balloons can be mechanically pressed against the catheter tube using a wire, such as suture or flossing wire. In some embodiments, a small amount of adhesive, such as cyanoacrylate, can be used to bond the wire to itself, avoiding the need for chemical bonding between balloon and catheter tube.

In some embodiments, the attachment of inner balloons to the internal region of the catheter tube can include bonding or gluing and can be implemented with suitable adhesives or other suitable means. In some embodiments, the attachment of inner balloons to the internal region of the catheter tube can include mechanical pressure affected by a metal tube fitted inside the lumen of the catheter, over the distal end of the balloon.

In some embodiments, he folded balloons determine the interface of the catheter with the cavity target tissue, and it may enable the use of relatively stiff plastic for the catheter shaft (tube), while the relatively soft material of the inner balloon provides better tolerability, leading to better patient compliance.

According to some embodiments, the connectable catheter system can advantageously enable sustained release administration of a therapeutic substance along and through the walls of the indwelling stent tube, in addition to administration of the substance to the target cavity. In some embodiments, the connectable catheter system can advantageously further optionally allow drainage of bodily fluids, such as, urine, through the stent tube walls, while enabling the flow of the therapeutic substance therethrough, to reach the target cavity. According to some embodiments, in order to achieve this, the stent wall includes holes/apertures/openings at its perimeter.

In some embodiments, the specially designed stent apertures enable drainage of bodily fluids such as, for example, urine, through the stent, while enabling instillation of substances (such as medications) through the stent with minimal spilling out of the stent during instillation. In some embodiments, the aperture dimensions and shape can determine the flow therethrough. For example, as further detailed below, the hole can be designed/formed to block the flow of high-viscosity liquids, while enabling capillary flow of urine.

According to some embodiments, when applied with the connectable catheter system, such aperture can also enable the flow of instilled substances, such as medications, through the stent walls. Various design parameters can be used to determine and enable the control over the flow-ability, flow direction and flow-rate out of the stent wall, through the specialized openings. Exemplary design parameters include such parameters as: viscosity of the instilled substance, flexibility of the stent wall, aperture diameter, aperture angle through the wall, and the like.

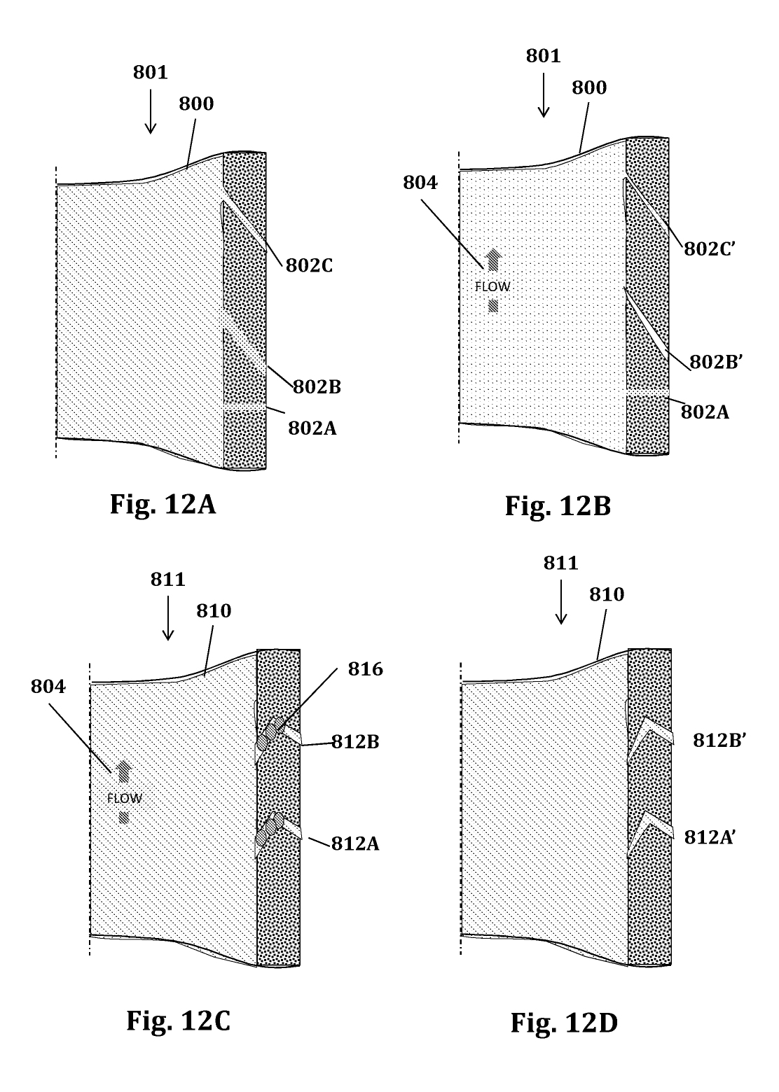

Reference is now made to FIGS. 12A-12D schematically illustrating a cross section view of internal wall of an indwelling stent having apertures in its lateral walls, according to some embodiments. As shown in FIG. 12A, wall (800) of stent (801), can include several types of apertures (802A-C) at its lateral walls. The apertures can traverse the walls, i.e., can form an inside-outside direct connection through the lateral walls of the stent. The apertures in the lateral wall may be similar, identical or different in shape, diameter and slant (angle relative to the flow direction). In some embodiments, the apertures are capillary sized. As shown in FIG. 12A, exemplary aperture 802A is designed/shaped to allow fluid passage based on the rheology of the fluid. For example, aperture 802A can be designed to block the flow of high-viscosity liquids (for example, medical substances instilled through the internal lumen of the stent), while enabling comparatively free flow of low viscosity fluids, such as urine. Apertures 802B-C, illustrated in FIG. 12A, are formed at a slanted negative angle, relative to the flow direction (Flow 804, shown in FIG. 12B). In such a setting, when a relatively viscous substance is administered and is flowing through the stent tube, the pressure formed by the flow of viscous substance, blocks slanted apertures (802B-C), thereby preventing leaks of the substance through the walls (from within the stent to the outside) during instillation and enabling flow of the substance through the length of the catheter system (up to the target cavity) after the instillation, when the flow pressure is released. This is illustrated in FIG. 12B, showing apertures 802B′-and 802C′ at a closed state (the inner opening assumes a conical, blocking shape). Such a setting can further allow capillary flow thought the apertures from outside to the inside of the stent tube. After administration of the substance (for example, by injection), the pressure inside the stent tube is decreasing. Consequently, the slanted apertures can now assume an open state, thereby enabling gradual elution of the substance via the apertures, along the length of the tube. Such advantageous setting, can allow sustained release of the therapeutic substance at designated regions along the catheter system tube, as determined by the position, size and angle of the apertures in the lateral walls. In some embodiments, the effect of the slanted apertures is further enhanced when the stent wall is flexible (for example, Shore A 30) and when the instilled liquid substance is viscous (for example, TC-3). The instilled viscous liquid applies pressure on the stent inner wall, which closes the aperture to a conical shape, throughout the duration of the administration (instillation) of the substance. At any other time, the apertures are open and can enable flow in and out of the stent.

As shown in FIG. 12C, wall (810) of stent (811), include apertures/opening (812A-B) at its lateral walls, wherein the apertures are double-slanted. As shown in FIG. 12C, apertures 812A-B are double slanted, by having a first part (starting at the internal region of the stent lateral wall) having a positive angle (relative to the substance flow (814) in the stent tube) within a portion (such as about half) of the lateral wall width, and a second, negative angle part (ending at the external region of the lateral stent wall). In such a setting, when a relatively viscous substance is administered and is flowing through the stent tube, the positive-angle aperture part is filled with the substance (816), which does not further flow outside. When the substance pressure flow ceases, the substance can gradually elute outside of the aperture, via the negative angle part. For example, instillation of viscous liquid (e.g., TC-3 sustained release gel mixed with medication) into the stent, will push the liquid into the aperture and partially fill the aperture volume. Thereafter, for a prolonged duration, the liquid will be intermittently exposed to urine and slowly dissolve—releasing the medication outside the stent, along its length. Such advantageous setting, can allow sustained release of the therapeutic substance at designated regions along the catheter system tube, as determined by the position, size and angles of the apertures in the lateral walls. In FIG. 12D, no substance is administered, and the double slanted apertures (812A′ and 812B′), maintain a closed position, whereby the positive angle part may be closed at a conical shape.

According to some embodiments, apertures in the lateral walls of a tube may be formed/shaped/created by various means. For example, highly accurate means for generating apertures (such as by laser drilling) can be used for making apertures in the walls of the tubes. As mentioned above, the apertures can assume various shapes, sizes, diameters, angles, and the like. For example, for making slanted apertures, the tube may be held/positioned at a desired angle when making the slanted apertures. For example, for making double slanted apertures, fine double-slanted apertures can be drilled into the stent wall at a positive angle to the flow direction, while connecting apertures are drilled in the opposite direction and the unusable parts of the drilled apertures can be sealed, for example, by glue beads.

According to some embodiments, the apertures in the lateral wall are optionally similar, identical or different in shape, diameter, and slant (angle relative to the flow direction). In some embodiments, the apertures are capillary sized. In some embodiments, the apertures may optionally be distributed evenly or non-evenly along the circumference of the stent walls and/or along the length of the walls. Each option is a separate embodiment.

According to some embodiments, there is provided a connectable catheter system, comprising: an intermediary catheter and a reconnectable indwelling stent comprising apertures at a lateral wall thereof, configured to allow extended release of therapeutic substances through the external walls of the stent, along at least a portion (length) thereof .

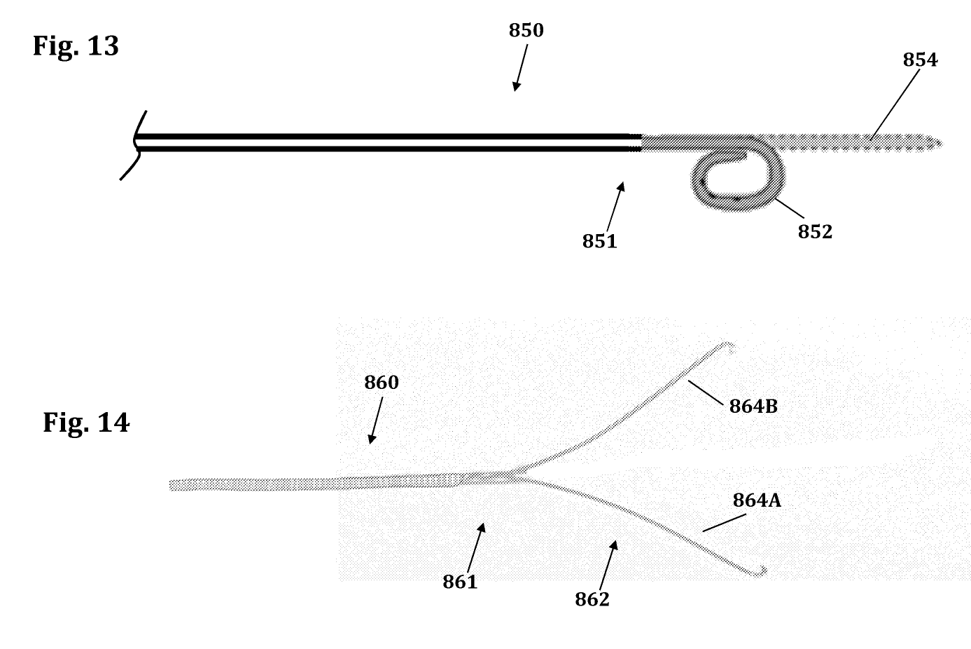

According to some embodiments, various means (members) may be used to anchor the catheter system elements, such as, the indwelling stent in internal target tissues, such as, internal cavities. The anchoring means (also referred to herein as anchoring members) may be reversible anchoring means that may assume a closed position (for example, when being inserted to the cavity) and an open position when being anchored in the cavity. Reference is now made to FIG. 13, which shows a target section of an indwelling stent, having an anchoring means, according to some embodiments. As shown in FIG. 13, stent 850 has at its target section end (851) an anchoring means (shown as “pig tail” anchoring mean, 852). The pig tail anchoring means can be made/formed as an integral part of the stent, by shaping the stent tip in one or more loops to the form of a “pigtail tip”. When in a loop form, the pig tail tip can be used to anchor the stent to its target area/cavity. The loop is designed to provide a predetermined resistivity to being pulled out of its position in regular use, and may further enabling intentional removal when needed, by acquiring a straitened position (854), which ablates the anchoring of the stent to the target region.

Reference is now made to FIG. 14, which shows a target section of an indwelling stent, having an anchoring means, according to some embodiments. As shown in FIG. 14, stent 860 has at its target section end (861) an anchoring means (862). The anchoring means is shown in an open position, having two flexible arms (864A-B), spread open, allowing anchoring of the stent to its location. According to some embodiments, such foldable anchoring means is inserted (for example, into the ureter) when in closed position and can assume an openposition inside the cavity. In some embodiments, such foldable anchoring means can be made of a memory material. For example, the foldable anchoring means can be made of memory metal, designed to be small enough to enable its passage in the tract, where the catheter is used as well as plastic members of suitable mechanical properties (such as flexibility). In some embodiments, the memory shape tip can be made of flexible material that is held in its place during insertion for example, by a retractable protective sleeve that can be removed when the member is positioned at the desired location. In some embodiments, the flexibility and shape of the memory shaped tip can be designed for a variety of applications, such as, for example, retention in the kidney, and or to provide a platform for supporting the use of various additional funcationla elements, such as, direct or indirect heating elements, as detailed below.

According to some embodiments, the connectable catheter system disclosed herein can advantageously allow heating of substances administered therethrough and/or heating of internal target organs/cavities. In some embodiments, heating of the target cavity and/or the administered substance enhances treatment efficiency and efficacy, based on the treated organ and the administered substances. In some embodiments, heat can be generated and transferred into the target cavity by means, such as, but not limited to: electrical heating, chemical heating, microwave heating, pressure waves (such as High-Intensity-Focused-Ultrasound (HIFU)) heating, heated liquids (such as water), infrared energy heating (that may be affected by either external infrared radiation or by transfer of infrared energy via fibers or by energizing infrared sources, such as infrared LED that can mounted at the tip of the indwelling stent), and the like. In some embodiments, a heat generating element (unit/source) may be responsible for generating the heat (energy required for the heat) and/or controlling the heat parameters (i.e., heating temperature, duration of heating, time to reach target temperature, etc.). In some embodiments, the heat parameters may be controlled by a control unit that may be either independent or part of the heat generating element (unit).

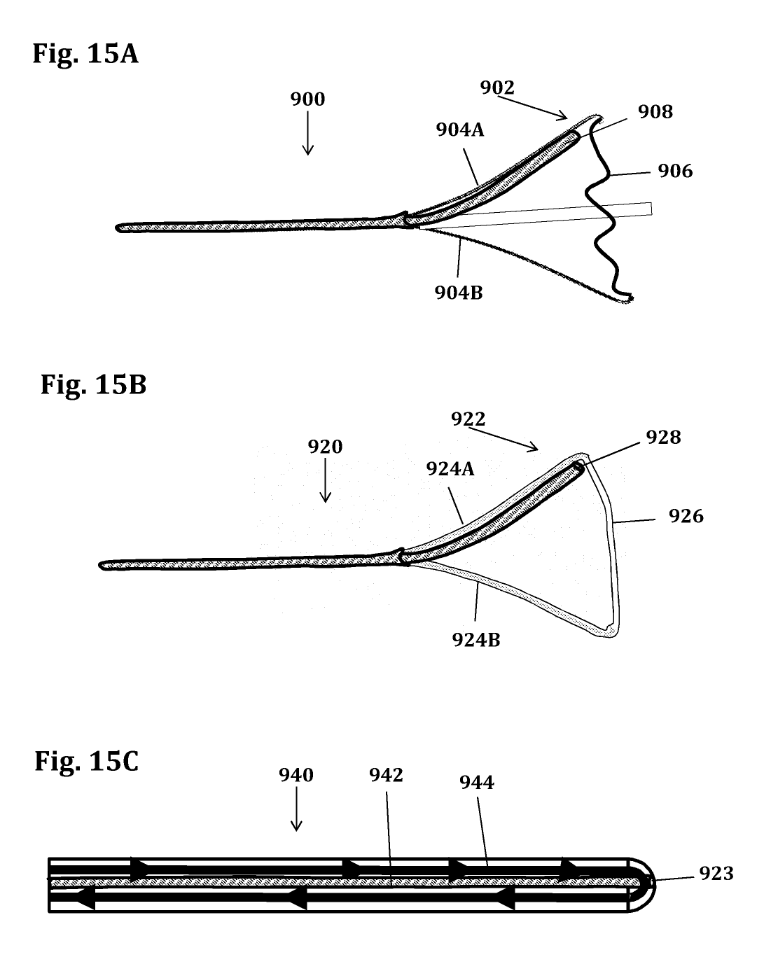

Reference is now made to FIGS. 15A-C, which schematically show target section of an indwelling stent, having heating mean elements. As shown in FIG. 15A, stent 900 has at its target section end, an anchoring element, 902, which has two flexible arms, 904A-B, shown in their open state. Further shown is heating conductor element, 906, which connects between the arms of the anchoring element. The heating conductor element can be, for example, an electrical resistivity load or a high frequency antenna, designed to impart the energy required to heat the cavity area from the normal body heat to a desired target temperature (such as, for example, in the range of 40 to 46° C., as required. For example, the target temperature is 44° C.). In such settings, the anchoring means may be made of memory metal. The heating capability coexist with other functionality of the catheter, such as substance administration into the cavity, for example, via instillation conduit, 908.

Reference is now made to FIG. 15B, which schematically shows stent 920, having at its target section end an anchoring element (922), which has two flexible arms, 924A-B, shown in their open state. Further shown is heating fluid conduit, 926, which is located around the external perimeters of the arms of the anchoring elements, and further extends along the internal lumen of the stent to connect to a heating fluid source, capable of providing, and circulating via the dedicated heating fluid conduit, a suitable heating fluid (such as water), heated to a desired temperature. By providing such circulation of heating fluid, the cavity, in which the stent end is anchored can be heated to a desired temperature, which can be maintained to any period of time. The heating capability coexist with other functionality of the catheter, such as substance administration into the cavity, for example, via instillation conduit, 928.

Reference is now made to FIG. 15C, which schematically illustrate yet another embodiment of heating a target cavity using an indwelling stent. Shown in FIG. 15C is the target section end of stent 940. Further shown are two separable conduits located within the stent lumen: an instillation conduit, 942, capable of providing substances (such as, therapeutic medication to the target cavity (wherein the substance can be eluted from the conduit to the target cavity via instillation opening, 943); and a heating fluid conduit (944), capable of providing, from an external source, and circulating via the dedicated conduit, a suitable heating fluid (such as water), heated to a desired temperature. By providing such circulation of heating fluid, the cavity, in which the stent end is located can be heated to a desired temperature and moreover, maintain the desired temperature to any determined period of time.

According to some embodiments, heating by a direct heating member (for example, an electrical resistivity member, water conduit, etc.) may impose problematic side effects, since heat is highest next to the heating member and is reduced considerably further away. To this aim, the direct heating may be combined, in some embodiments with administration of a gel (such as, RTGel), which is made of high water percentage (such as over 70% water), thereby the heat is conveyed more efficiently, and enhances the use of direct heating.

According to some embodiments, the use of a connectable catheter system utilizing directional connection enabling several, non-centric connections and optionally cavity anchoring (for example, using with memory shaped tips) can advantageously enable precise and controlled heating of an internal cavity, by circulation of heated liquid (such as, water) through the connectable catheter system.

In some embodiments, the heated liquid may be transferred through a dedicated conduit (such as a thin tube) from outside the body, through continuous conduits formed at both parts of the catheter system, to an internal tube (such as a flexible tube), inside the cavity to be heated, that is held in its place by a suitable anchoring member.

In some embodiments, in order to maintain the higher-than-body-temperature of the heating liquid until it reaches its target in the cavity, the system should preferably provide some insulation along the path; and/or b) enable fast, quick and efficient liquid transfer. Accordingly, the unique and advantageous design of the connectable catheter system, utilizing directional connections, enables using more than a single conduit for fluid transfer through the catheter system.

In some embodiments, in order for the tube to lose heat in the target cavity area, the length and shape of the liquid tube can be designed for maximal exposure by making the tube path longer that the shortest path between the anchor members, so as to provide improved control over consistency and accuracy of heating.

In some exemplary embodiments, the connectable catheter system may be used for renal cavity heating. The urinary system includes two kidneys that produce urine, the ureters that pass urine from each kidney into the bladder in the lower abdomen that stores it. When the bladder is full, the muscles in the bladder wall tighten to perform urination. Urine leaves the bladder out of the body through the urethra. The inner lining of the urinary system is composed of several layers of transitional cells. Carcinoma of the Transitional Cells (TCC) starts in the epithelial cells of the urinary system. Flushing with chemotherapeutic substances is intended to destroy cancer cells or cells that have a high malignant potential, either independently or after surgical removal of the cancerous tissue. Combining local heat and use of chemotherapy substances, such as Mitomycin C (MMC) has been shown to affect superficial bladder cancer better than using MMC alone.

Accordingly, in some embodiments, there is provided a system and method to combine repeated hyperthermia plus MMC treatments of the upper urinary tract, to treat cancer is a subject in need thereof. The system for combining hyperthermia with MitoGel, which contains Mitomycin C, includes the connectbale cathther system, a heat generating element and a therapeutic substance, such as, MitoGel . In some embodiments, heat can be generated and transferred into the upper urinary system by means of electrical heating, chemical heating, microwave heating, infrared heating, radio frequency (RF) heating, pressure waves (such as High-Intensity-Focused-Ultrasound), heated fluid (such as, water), or the like, or any combination thereof. In some embodiments, the heat generating element is responsible for generating, controlling and/or maintaining the heat. Each possibility is a separate embodiment.

In some embodiments, when used in the ureteral system, the indwelling stent may further include a distal balloon configured for temporal blocking of the ureteropelvic junction (UPJ). Utilizing such as balloon may be used to generate UJP blockages at the point where the kidney attaches to one of the ureters, thereby inhibiting the flow of liquid such as urine out of the kidney. Using such UPJ balloon can aid in maintaining fluids (such as, therapeutic substances) in the renal cavity. In some embodiments, the indwelling stent may thus include at least two separable lumens and an inflatable balloon configured for temporal blocking of the UPJ, for circulating liquids in the renal cavity. Such liquids may be heated to 44° C. and may contain drugs such as chemotherapeutic drugs that are known to have an improved medical effect on heated cancerous tissue. In some embodiments, the liquid may be circulated at a known volume or by monitoring the liquid volume in the renal cavity via X-Ray imaging or by monitoring the excreted liquid volume. In some embodiments, the indwelling catheter may further include a tip with a pre-shaped anchoring member for anchoring the stent in the renal pelvis, allowing heated liquid to circulate in the renal cavity.

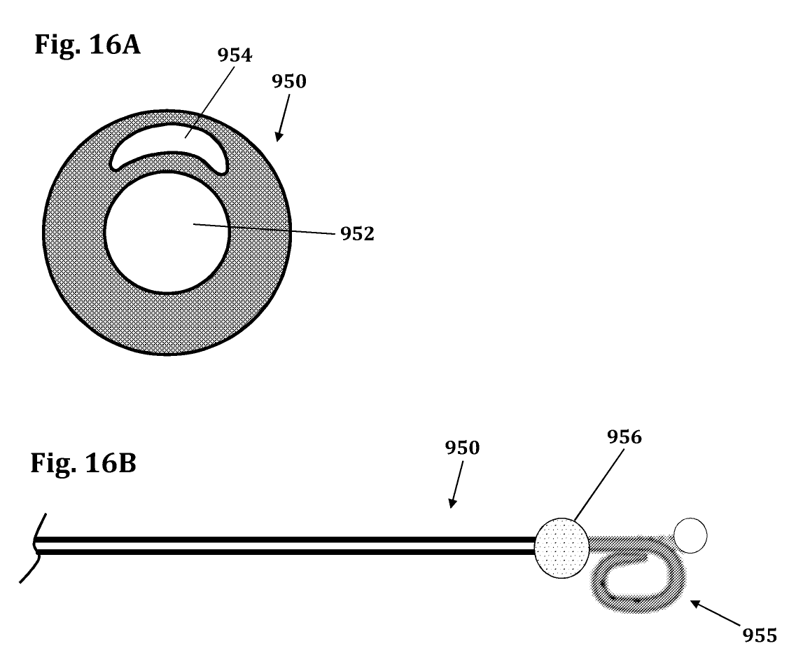

Reference is now made to FIGS. 16A-B, which show schematic illustrations of a target section of an indwelling stent having two internal conduits and a balloon for ureteropelvic junction (UPJ) for urine blockage. FIG. 16A illustrates a cross section top view of stent 950, having two separable internal lumens/conduits, including main lumen (952) and inflation lumen (954). The main lumen is configured to allow fluid passage of substances (such as, medication) administered to the target region. The inflating lumen is configured to provide fluid passage (such as, air), to inflate a UPJ balloon, located at the target end of the stent. FIG. 16B show a perspective illustration of stent 950, having at its target end an anchoring mean (exemplary shown in the form of a pig-tail anchoring mean, 955) and a UPJ balloon (956), shown in its inflated state. In such a setting, when the stent end is placed and anchored via the anchoring means in the target cavity (kidney), the balloon is positioned such that a ureteropelvic junction (UPJ) blockade is formed at the region where part of the kidney attached to one of the ureters, thereby inhibiting the flow of liquids (such as urine) out of the kidney, to result in longer period of time, in which the therapeutic substance, administered via the catheter system reside in the target cavity. This can results in improved treatment efficacy. In some embodiments, the UPJ blockade can be utilized to enable filling essentially the kidney with liquid medication, (which can include, for example a chemotherapy agent such as mitomycin-C), where the medication is heated to a temperature that is known to positively affect the absorption of the chemotherapy agent into the kidney tissue, thereby improving the therapeutic effect of the medication. In some embodiments, absorption of the medication can be improved by pre-flushing the cavity with beneficial chemical agents, such as salicylic acid, prior to filling the cavity (for example, kidney) with a suitable medication.

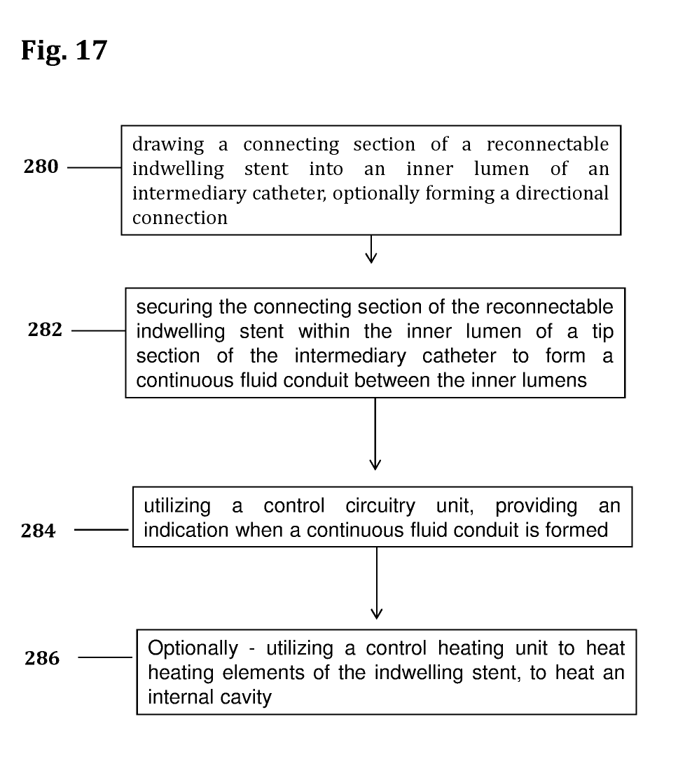

Reference is now made to FIG. 17, which is a block diagram of a method of connecting a connectable catheter system, according to some embodiments. As shown in FIG. 17, at step 280, a connecting section of the indwelling stent (located within the subject body) is drawn into the inner lumen of the intermediary catheter. Optionally, directional connections are formed, as disclosed above herein. Next, at step 282, the connecting section of the indwelling stent is secured within the inner lumen of a tip section of the intermediary catheter, while sealing the connection, to form a continuous fluid conduit between the inner lumens of the intermediary catheter and the intermediary catheter. Optionally, further internal lumens/conduits are formed. At step 284, a control circuitry unit is utilized, to provide an indication when the continuous fluid conduit is formed, to allow the use of the connectable catheter system for the transfer of fluids and other materials to and from an external body location to an internal body location. At optional step 286, a heating circuitry unit (heat generating element) may be utilized, to energize or heat the heating elements of the indwelling stent, designed to heat the target cavity.

In some embodiments, securing the connecting section of the indwelling stent within the inner lumen of the tip section of connectable catheter includes deploying a securing member located at an inner lumen of the connectable catheter.

In some embodiments, drawing a connecting section of an indwelling stent into an inner lumen of the connectable catheter is conducted by inserting a stylet into the inner lumen of the connectable catheter, the stylet comprises, at a tip section thereof, an attachment member configured to attach to the connecting section of the catheter and, such that when the stylet is proximally retracted within the inner lumen of the connectable catheter, the indwelling stent is pulled into the inner lumen of the connectable catheter.

In some embodiments, the method may further include automatically providing, utilizing an indication unit, an indication when a connection between the attachment member of the stylet and the connecting section of the indwelling stent is formed.

According to some embodiments, once a fluid-tight connection (conduit) has been formed between the respective ends of the indwelling stent and the intermediary catheter, a material of choice (such as, but not limited to, a medication or other composition) that is to be instilled via the continuous catheter thus formed, may be attached to a connector at the external section of the intermediary catheter. For example, the medication or composition may be contained in a syringe which is attached to a respective connector.

The material to be instilled into the target organ/cavity may be delivered in any formulation or compositions and may include various active or non-active ingredients, depending on the target organ and the required treatment regime. In some embodiments, the composition of choice may be delivered in the form of a gel that solidifies in the body and slowly releases the drug over a period of time, such as several hours. In some exemplary embodiments, the medicated gel has a low viscosity at below body temperature and high viscosity at body temperature. Such a gel can be instilled, for example, into the upper urinary tract in its fluid state and then become very viscous as its temperature rises to body temperature in the upper urinary tract. Using this type of gel enables the filling of essentially the entire volume of the upper tract (or any target cavity to be treated), including difficult to reach areas, such as renal calyces and the complete length of the ureter.

In some non-limiting examples, liquid medication, for example chemotherapy mixed with water for injection may be instilled into the target organ. In some exemplary embodiments, the medication is mitomycin C.

In another non-limiting example, liquid medication, for example chemotherapy mixed with water for injection is instilled into the renal pelvis while fluids are extracted from bladder. In a non-limiting example, the rate of instillation and extraction are similar to form a closed circulation of medication throughout the urinary tract. In a non-limiting example, a heater (heating member or heat generating source) may be integrated into the medication closed circulation to provide heated medication into the urinary tract. In some embodiments, the heater is an external heater member. In some embodiments, the heater is an internal heater member. In some embodiments, the heater may comprise a combination of external and/or internal heater members. In some embodiments, the medication is in a form of a gel. In some embodiments, the gel comprises therapeutic substances. In some embodiments, the gel comprises mitomycin C.

In some embodiments, it is possible to use the connection to the indwelling stent to introduce medications to address potential adverse effects that are known to effect indwelling stents. Such medication can include pain-relief (such as lidocaine), infection drugs (such as antibiotics) and similar This capability enhances the safety and reduces medical risks of indwelling stents of the present invention.

According to some embodiments, the systems, devices and method disclosed herein may be used for administration of various substances to various internal target organs, while minimizing risks associated therewith (such as radiation hazards, insertion trauma, anesthesia complications, infections, irritation, sensitizations, injuries, and the like.).

According to some embodiments, the systems and methods disclosed herein may be used for the treatment of various conditions.

In some embodiments, there is provided a method of treating cancer. In some embodiments, the cancer is urinary tract cancer. In some embodiments, the cancer is bladder cancer. In some embodiments, the cancer is kidney cancer. In some embodiments, the cancer is urethral cancer. In some embodiments, the cancer is a body cavity cancer.

In some embodiments, the system may be used to provide a medication to a target organ, in order to treat a condition, such as cancer.

In some embodiments, the target organ may be selected from, but not limited to: stomach, bladder, kidney, heart, intestines, uterus, lungs, urinary tract, and the like, or combinations thereof. Each possibility is a separate embodiment.

In some exemplary embodiments, the system disclosed herein may be used for instillation of composition to the urinary tract.

In some exemplary embodiments, the system disclosed herein may be used for instillation of heated composition to the urinary tract.

In some exemplary embodiments, the system disclosed herein may be used for instillation of composition to the urinary tract and provide heat circulation of the compositions and/or tissues of the urinary tract.

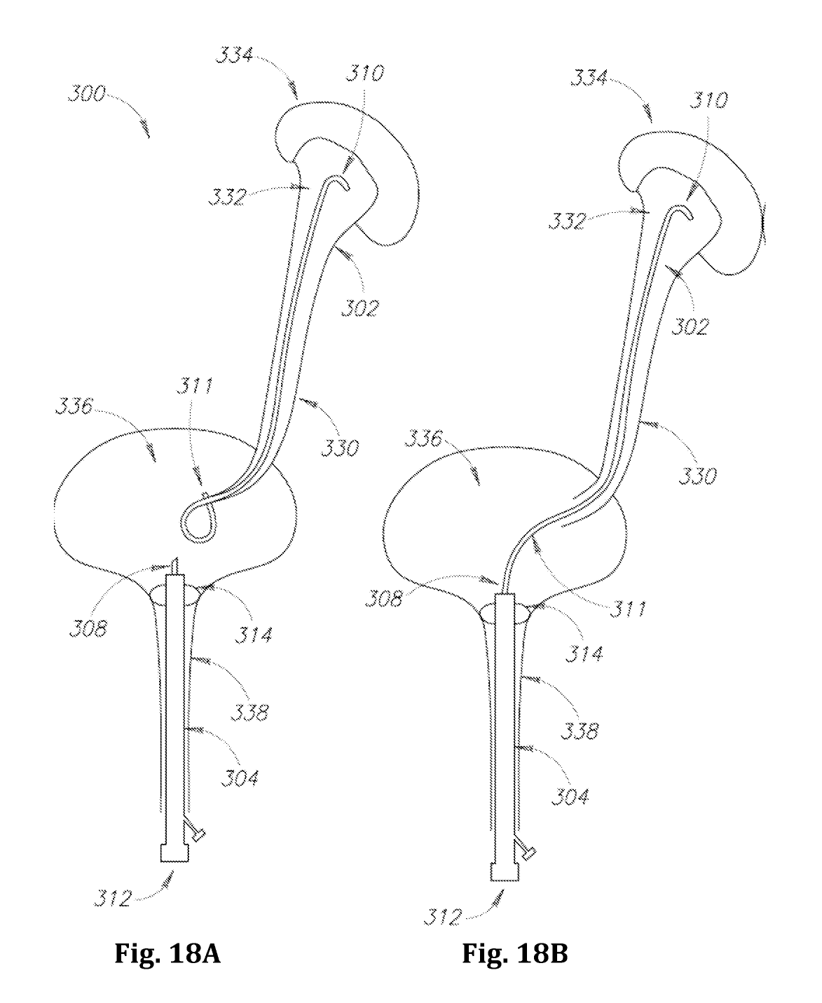

Reference is now made to FIGS. 18A-B, which show a catheter system (300) after being positioned in a urinary tract. Shown in FIG. 18A is indwelling stent (302) after being positioned in a ureter (330) of a male or female subject. The target section (310) of indwelling stent (302) is positioned in renal pelvis (332) of a kidney. The connecting section (311) of indwelling stent (302) is positioned in the urinary bladder (336) of the subject. The target section (310) of the indwelling stent (302) may be able to coil into a “pigtail” or “double pigtail”, in order to anchor the target section (310) of indwelling stent (302) in the renal pelvis (332) and prevent migration from the renal pelvis (332) downwards or upwards. The indwelling stent (302) may comprise one or more anchors to prevent migration thereof. The intermediary catheter (304) is adapted/configured to be inserted through the meatus into the urethra until the tip section (308) is located inside bladder (336). As shown, intermediary catheter (304) is positioned in a urethra (338) of the subject with the tip section (308) being positioned in bladder (336), and the external section (312) of the being positioned outside the body. After inflation of anchoring balloon (314) inside bladder (336), the intermediary catheter (304) may be pulled, such that anchoring balloon (314) is located in the neck region of bladder (336). The tip section (308) of the intermediary catheter (304) is configured to reversibly connect to the connecting section (311) of indwelling stent (302) inside bladder (336), as shown in FIG. 18B. When connected, the indwelling stent (302) and the intermediate catheter (304) form a continuous channel from outside the body to the renal pelvis (target organ). As detailed above, approximation of the respective ends of the intermediary catheter and indwelling stents inside the bladder may be facilitated by various means, such as, use of magnets (for example, hollow cylindrical magnetic tips of opposite polarities, external magnets, and the like). For example, in some cases, when the bladder sags and the connecting section of the indwelling stent is located further from the meatus, it may be beneficial to bring the ends to closer proximity before the intermediary catheter scanning. This may be achieved, for example, by a magnet that is manipulated outside of the body together with magnetic or magnetizable particles on the respective tips of the respective catheters to bring the ends together. In some embodiments, the external magnet will be radially polarized so as to attract both magnet polarities.

When the respective ends of both the indwelling stent and the intermediary catheter are present/located in the bladder (as demonstrated in FIG. 18A), the ends may attract each other by the magnetic field and spontaneously form a stable, fluid tight connection between them (as demonstrated in FIG. 18B). As detailed above, means may be provided to ensure an end to end attachment of the components and prevent a side by side attachment.

According to some embodiments, when placed in the target region, heating of the target cavity may be commenced, by any of the means disclosed herein. In some embodiments, an internal UPJ balloon may be used to enhance treatment efficacy.

According to some exemplary embodiments, when used in the urethra system, the outer diameter of the indwelling stent may be for example, 6F from the target (renal) end and all along the part that is deployed in the ureter. This would allow the indwelling stent to be inserted, as a non-limiting example, over, a 0.038″guide wire. The indwelling stent may be flared at its bladder end so as to terminate at the bladder end with an outer diameter, for example, of 16 F. In some embodiments, the opening of the indwelling stent at the target section may allow instillation of a high viscosity gel to the renal pelvis.

According to some embodiments, the size and diameter of the indwelling stents and/or the intermediary catheter may vary according to the target organ and the internal location thereof. In some exemplary embodiments, the indwelling catheter may have a length of about 35-40 cm (for a male subject), and about 25-30 cm (for a female subject). For example, the indwelling stent may have a length of about 26-34 cm (for Male or Female subjects), and about 20-24 cm (pediatrics). For example, a guide rod (for example, a mandrel) may have a length of 40-45 cm (for male), and 30-35 cm for female. For example, the stylet may be at a length of about 40-50 cm.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

### EXAMPLES

### Example 1

### Using a Connectable Catheter System for Heating the Renal Cavity by Hot Fluid Circulation

To heat the intended renal cavity, a multi-lumen ureteral stent is used. The stent includes: a central lumen designed for transfer of fluid to the target cavity; b) an anchoring member in its distal (target) end. In this example, the anchoring member is closed in a sheath sleeve and can be opened by being pushed out of the sheath; c) a thin and flexible tube that connects between two of the stent lumens and is held by the sheathed anchoring member; and a diametrical magnet at the proximal end (connecting end) that is configured to accommodate conduit connection to the corresponding stent lumens.

The stent is inserted through the urinary urethra into the bladder and through the ureteral orifice and the ureter—to reach the kidney. The insertion is performed following standard endourology techniques, including insertion of a guidewire and threading the stent with a pushing member over the guidewire till it reaches a location such that the distal end of the stent is in the renal pelvis and the proximal end with the magnet is in the bladder. That location is verified by online medical imaging. After the stent is located as determined by a physician, the anchoring member is pushed out of its sheath and opens in the renal pelvis and the pushing member is extracted from the body.

With the stent in place and at the decision of the physician—an intermediate catheter that is equipped with an anchoring balloon, is inserted through the urinary urethra into the bladder. That catheter's balloon is inflated when positioned in the bladder to enable inflating the bladder by adding liquid such as saline in order to minimize tissue pleats and wrinkles while essentially preventing the liquid voiding through the urethra.

A stylet is then inserted through the intermediate catheter, such that is designed with multi-lumens and is equipped with a diametrical magnet at the distal end that is built to accommodate conduit connections and fit the stent magnet. By moving the stylet back and forth inside the bladder, the magnets on the stylet and stent are brought into proximity and attach to one another. The diametrical magnetic forces cause the attachment to occur according to the designed fit between conduits, to ensure alignment thereof, such that: a) liquid (including cooled and medicated gel, MitoGel), can be inserted through the stylet and stent to the renal pelvis; and b) heated liquid can be circulated through a conduit that includes a lumen in the stylet, conduits in both stylet and stent magnets, lumen in the stent, the flexible tube held by the stent anchoring member and back in a separate but similar, parallel, conduit.

The passage of heated liquid through the flexible tube results in heating of the renal pelvis. If the renal pelvis and kidney are essentially filled with water-based medicated gel—such as MitoGel—the heat is transferred throughout the gel and the kidney with minimal heat loss. The amount of heat that is transferred through the flexible tube depends mainly on the length of the flexible tube and the insulation provided by the tube wall and can be controlled by liquid input temperature and circulation flow rate.

After the treatment is finished, the stylet can be disconnected from the stent by pushing the magnets apart and the catheter can be removed from the bladder after its balloon is deflated—leaving the stent in its position. The treatment can be repeated as needed by inserting a new catheter and stylet as described above, and insertion of medicated gel and circulating heated liquid.

### Example 2

### Using a Catheter System for Heating the Renal Pelvis by Hot Water Circulation

To heat the intended renal pelvis and kidney, a continuous ureteral catheter is equipped with a central lumen, an anchoring member and a flexible tube (similar to the stent arrangement of Example 1). This continuous catheter can be inserted through the urinary tract to the kidney and enable the heated liquid circulation, via the heating fluid conduit, with no other external connecting parts (such as the stylet used in Example 1).

What is claimed is:

1. A connectable catheter system for providing extended release of a substance in a target cavity, the system comprising:
an intermediary catheter comprising an external section and a tip section, the tip section is configured to be inserted into a body of a subject; and
a reconnectable indwelling stent comprising a connecting section and a target section, the target section being configured to be located within a body of the subject, wherein the indwelling stent comprises at lateral walls thereof, apertures configured to allow extended release of a substance administered therethrough;
wherein the connecting section of the reconnectable indwelling stent is configured to reversibly connect, within the body of the subject, to the tip section of the intermediary catheter to form a continuous conduit between the intermediary catheter and the reconnectable indwelling stent.

2. The connectable catheter system of claim 1, wherein the connecting section of the reconnectable indwelling stent is configured to fit into an inner lumen of the tip section of the intermediary catheter.

3. The connectable catheter system of claim 1, wherein the apertures traverse the outer walls of the indwelling stent to allow release of substances along the length of the indwelling stent.

4. (canceled)

5. The connectable catheter system of claim 1, wherein the apertures are capillary sized.

6. The connectable catheter system of claim 1, wherein the apertures are perpendicular to the flow of the substance in an internal conduit of the indwelling stent.

7. The connectable catheter system of claim 1, wherein the apertures are slanted at a negative angle relative to the flow of the substance in an internal conduit of the indwelling stent.

8. The connectable catheter system of claim 1, wherein the apertures are bi-slanted.

9.-11. (canceled)

12. A connectable catheter system for delivering a substance to a kidney of a subject, the system comprising:
an intermediary catheter comprising an external section and a tip section, the tip section is configured to be inserted into a body of a subject; and
a reconnectable indwelling stent comprising a connecting section and a target section, the target section being configured to be located within a kidney of a subject, wherein the indwelling stent comprises at the target section an anchoring member and an inflatable ureteropelvic junction (UPJ) balloon;
wherein the connecting section of the reconnectable indwelling stent is configured to reversibly connect, within the body of the subject, to the tip section of the intermediary catheter to form a continuous conduit between the intermediary catheter and the reconnectable indwelling stent, and wherein the anchoring member is configured to anchor the indwelling stent within the kidney.

13. The connectable catheter system of claim 12, wherein the anchoring member is selected from a pig-tail anchoring member and a memory shape tip anchoring member.

14. The connectable catheter system of claim 12, wherein the indwelling stent comprises more than one internal conduits.

15. The connectable catheter system of claim 14, wherein an internal conduit of the indwelling stent is configured to provide fluid to inflate the UPJ; wherein upon inflation of the UPJ balloon, blockade of the ureteropelvic junction is initiated.

16.-19. (canceled)

20. The connectable catheter system of claim 12, further comprising one or more heating elements configured to heat the substance and/or internal tissues.

21. A connectable catheter system for heating an internal target tissue of a subject, the system comprising:
an intermediary catheter comprising an external section and a tip section, the tip section is configured to be inserted into a body of a subject; and
a reconnectable indwelling stent comprising a connecting section and a target section, the target section being configured to be located within the body of the subject, at a target tissue, said indwelling stent comprises heating elements;
wherein the connecting section of the reconnectable indwelling stent is configured to reversibly connect, within the body of the subject, to the tip section of the intermediary catheter to form a continuous conduit between the intermediary catheter and the reconnectable indwelling stent, and wherein the heating elements of the indwelling stent are configured to heat the internal target tissue of the subject.

22. The connectable catheter system of claim 21, wherein the indwelling stent comprises more than one internal conduits.

23. The connectable catheter system of claim 21, comprising forming directional connections between the intermediary catheter and indwelling stent utilizing diametrical magnets to form additional internal dedicated continuous conduits.

24. The connectable catheter system of claim 21, wherein the heating element is selected from a conductive element, a fluid heating element, an energy source and an infrared light emitting diode (LED).

25. The connectable catheter system of claim 21, further comprising an external heat generating element wherein the external heat generating element is configured to transfer said heat or energy through a dedicated conduit to the heating element of the indwelling stent, to thereby allow the heating element to heat the target tissue.

26. (canceled)

27. The connectable catheter system of claim 25, wherein a heat or energy source comprises microwave heating, radiofrequency heating, electrical heating, chemical heating, infrared heating, ultrasound heating, fluid heating, or combinations thereof.

28. The connectable catheter system of claim 21, providing circulation of heat in the target cavity.

29. (canceled)

30. The connectable catheter system of claim 21, wherein the indwelling stent further comprises an anchoring member, configured to anchor the target section of the indwelling stent in the target tissue.

31.-34. (canceled)
